(19) United States
(12) Patent Application Publication
(10) Pub. No.: US 2025/0297233 A1
(43) Pub. Date: Sep. 25, 2025

(54) GENETICALLY ENCODED SYSTEMS FOR GENERATING OXYGEN IN LIVING EUKARYOTIC CELLS

(71) Applicant: The General Hospital Corporation, Boston, MA (US)

(72) Inventor: Vamsi K. Mootha, Cambridge, MA (US)

(21) Appl. No.: 18/860,518

(22) PCT Filed: Apr. 25, 2023
(86) PCT No.: PCT/US2023/019755

§ 371 Date: Oct. 25, 2024

Related U.S. Application Data
(60) Provisional application No. 63/335,210, filed on Apr. 26, 2022.
(60) Provisional application No. 63/411,388, filed on Sep. 29, 2022.

(51) Int. Cl. C12N 9/02; C12N 5/09; C12N 15/86

(52) U.S. Cl. CPC C12N 9/0069; C12N 5/0693; C12N 15/86; C12Y 113/11049; C12N 2740/15043

(57) ABSTRACT

Described herein are compositions and methods for generating oxygen in living eukaryotic cells, e.g., animal cells, by expressing a Cld enzyme (i.e., chlorite dismutase, chlorite O₂-lyase, chlorite:O2 lyase), optionally in combination with a transporter, in the cells.

### CLAIM OF PRIORITY

This application claims the benefit of U.S. Provisional Application Ser. No. 63/335,210, filed on Apr. 26, 2022 and U.S. Provisional Application Ser. No. 63/411,388, filed on Sep. 29, 2022. The entire contents of the foregoing are incorporated herein by reference.

### TECHNICAL FIELD

Described herein are compositions and methods for generating oxygen in living eukaryotic cells, e.g., animal cells, by expressing chlorite dismutase (Cld; also called chlorite O2-lyase and chlorite: O2 lyase) in the cells.

### BACKGROUND

Oxygen is vital for all forms of life and is one of the most widely used substrates in all of biochemistry (Raymond and Segre 2006). One of the most important events for life on our planet was the great oxygenation event (GOE), some 2.1-2.4 billion years ago (Lyons 2014), which changed our environment and spawned aerobic life on our planet. Oxygen provides a thermodynamically favorable terminal electron acceptor that helps to power metabolism and has been proposed as a pre-requisite for the emergence of complex forms of animal life (Nursall 1959). Since oxygen is a di-radical and can be toxic, numerous mechanisms evolved to allow organisms to safely wield its thermodynamic potential (Lu and Imlay 2021). In addition, oxygen plays a key role in signaling (Kaelin and Ratcliff 2018; Semenza 2012) and contributes to cell differentiation and development (Simon and Keith 2008). Humans have an absolute requirement for oxygen, only able to survive minutes in complete anoxia. At the other extreme, hyperoxia can also be devastating, leading to seizures, pulmonary toxicity, and retinopathy.

### SUMMARY

Oxygen is one of the most important molecules that has enabled life on our planet. In the research, technological, and medical arenas, there are few if any ways to manipulate oxygen in living cells or organisms with high spatiotemporal control. This application is based at least in part on the surprising discovery involving a genetic strategy for generating oxygen in living mammalian cells, e.g., human cells, by making use of an enzyme that converts chlorite into oxygen and chloride. This enzyme is abbreviated Cld and referred to as chlorite dismutase, chlorite O₂-lyase, chlorite:O2 lyase, chlorite lyase, and chlorite oxidoreductase in the scientific community, and the terms are used interchangeably (enzyme commission number EC 1.13.11.49). A Cld enzyme may be co-expressed in combination with a transporter. To our knowledge, this is the first system to system allows fine temporal and spatial control of oxygen production in animal cells, with immediate research applications.

Thus, provided herein are isolated eukaryotic cells expressing (i.e., engineered to express) a bacterial or archaeal chlorite dismutase (Cld). In some embodiments, Cld is connected to a targeting sequence, optionally wherein the targeting sequence directs the Cld to the mitochondria. In some embodiments, the Cld is expressed in the cytoplasm and the mitochondria. In some embodiments, the isolated cells also express (e.g., have been engineered to express) a chlorite transporter, e.g., an exogenous chlorite transporter. In some embodiments, the chlorite transporter is a sodium iodide symporter (NIS). In some embodiments, the NIS is encoded by SLC5A5, optionally comprising a sequence shown in Table 1. In some embodiments, the isolated cells are animal cells, e.g., mammalian cells, e.g., human cells, optionally CAR-T cells.

In some embodiments, the bacterial chlorite dismutase (Cld) is from Nitrospira defluvii (NdCld), Dechloromonas aromatica (DaCld), or Nitrobacter winogradskyi (NwCld). In some embodiments, the bacterial or archaeal chlorite dismutase (Cld) lacks a functional periplasmic targeting sequence.

Also provided herein are methods for generating oxygen in a eukaryotic cell, the method comprising culturing any of the cells described herein in a media comprising chlorite, e.g., 50 um to 5 mM chlorite, or at least 50, 70, 75, 100, 250, or 500 uM chlorite, or up to 1, 2.5, or 5 mM chlorite. In some embodiments, the chlorite is sodium chlorite. In some embodiments, the cell is viable in media comprising at least 1, 2.5, or 5 mM chlorite.

Also provided herein are transgenic non-human uni- or multi-cellular eukaryotic organism comprising a cell as described herein. In some embodiments, the organism is a worm or a mouse. Additionally provided are methods for generating oxygen in the transgenic non-human uni- or multi-cellular eukaryotic organisms, comprising maintaining the organism in an environment comprising chlorite,

In some embodiments, the chlorite is present at levels that would be toxic to a non-transgenic organism of the same species.

Additionally, provided herein are isolated Cld proteins that lack a functional periplasmic targeting sequence. In some embodiments, the Cld proteins comprise a sequence as disclosed herein, optionally without a tag (e.g., without FLAG) sequence. In some embodiments, a Cld protein is connected to targeting sequence to an organelle, such as the mitochondria. Also provided are nucleic acids comprising a sequence encoding any of the isolated Cld proteins, and optionally a sequence encoding a sodium iodide symporter (NIS). In some embodiments, the NIS is encoded by SLC5A5. In some embodiments, one or both of the sequences are codon optimized for expression in a eukaryotic cell, e.g., an animal cell, e.g., a human cell. In some embodiments, the sequences encoding a Cld and a transporter, (e.g., NIS) are located on a single nucleic acid. In some embodiments, a ribosomal skip sequence can be used between the Cld and NIS. In some embodiments, the ribosome skip sequence is a “2A” skip sequence, e.g., T2A, a P2A, an E2A, or an F2A; see, e.g., Liu Z, et al. (2017) “Systematic comparison of 2A peptides for cloning multi-genes in a polycistronic vector” Scientific Reports 7:2193. Also described herein are vectors comprising any of the nucleic acids, optionally a bi-cistronic vector that encodes both the Cld and the NIS, for expression of both.

Further provided are host cells comprising the nucleic acids and/or vectors as described herein, and optionally expressing the Cld and/or NIS proteins. In some embodiments, the host cell is an animal cell, e.g., a mammalian cell, e.g., a human cell. In some embodiments, the bacterial chlorite dismutase (Cld) is from Nitrospira defluvii (NdCld), Dechloromonas aromatica (DaCld), or Nitrobacter winogradskyi (NwCld). In some embodiments, the bacterial chlorite dismutase (Cld) lacks a functional periplasmic targeting sequence. In some embodiments, the host cell also expresses a sodium iodide symporter (NIS). In some embodiments, the NIS is encoded by SLC5A5, optionally comprising a sequence shown in Table 1.

Additionally, provided herein are methods for generating oxygen in a eukaryotic cell, comprising culturing any one or more of the host cells described herein. In some embodiments, the culturing is in a media comprising 50 μm to 5 mM chlorite, or at least 50, 70, 75, 100, 250, or 500 UM chlorite, or up to 1, 2.5, or 5 mM chlorite.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A-1C: Screening Cld variants for expression in human cells. 1A. Reaction catalyzed by the Cld enzymes. 1B. Structures of Cld enzymes from Nd (Lineage I) and Nw (Lineage II) (PDB accession #3NN2 and 3QP1; N- and C-termini are represented with green and red spheres, respectively). 1C. Lysates from HeLa cells transduced with lentivirus for indicated constructs were subjected to SDS-PAGE and immunoblotted to confirm expression of FLAG-tagged Cld variants, GFP, or loading control.

FIG. 2A-2E: Cld expressed in human cells assembles properly with high activity. 2A. Size exclusion chromatography profile of purified NdCld. 2B. SDS-PAGE analysis of purified NdCld visualized with coomassie. 2C. Absorption spectra of purified NdCld in the presence of 500 uM ferricyanide with (reduced) or without (oxidized) 2.5 mM dithionite. 2D. Time traces of molecular oxygen formation with different chlorite concentrations. 2E. Steady state kinetics of NdCld catalyzed oxygen production. Points represent average of four measurements and error bars show the standard error of the mean. Data were normalized to represent activity per 100,000 cells.

FIG. 3A-3D: Three-day toxicity of sodium chlorite to human HeLa cells. HeLa cells expressing (3A) GFP, (3B) NdCld, (3C) GFP+NIS, or (3D) NdCld+NIS were grown for three days with the indicated concentration of freshly prepared sodium chlorite. Cell counts and viability were assessed after 3 days of growth using a Vi-Cell BLU Cell Viability Analyzer. Shown is the mean+/−s.d. of triplicate measurements.

FIG. 4A-4E. On-demand generation of oxygen using Supplemental Oxygen Released from ChLorite (SNORCL) in human cells. The Agilent Seahorse XFe96 system was used to measure oxygen levels and oxygen consumption rates (OCR) in live, intact HeLa cells. 4A. Overview of the SNORCL system and reaction catalyzed by Cld and transport facilitated by NIS. 4B. Western blot of FLAG-NdCld in HeLa cells. 4C. Seahorse intact cell oxygen consumption rate measurements at 1% ambient oxygen with sequential additions of piericidin+antimycin (1 uM each) and sodium chlorite (0 (black, circles), 1 mM (dark grey, triangles), or 5 mM (light grey, squares), as also shown in the key for 4D) in Hela cells expressing GFP or NdCld. 4D. Seahorse intact cell oxygen consumption rate measurements at 1% ambient oxygen with sequential additions of piericidin+antimycin (1 uM each) and sodium chlorite (0, 1 mM, 2.5 mM, or 5 mM) in HeLa cells expressing GFP+NIS or NdCld+NIS. 4E. Traces of the average oxygen levels within two minutes upon sodium chlorite addition (black arrow) in the Seahorse experiments shown in FIG. 4C-D. OCR: oxygen consumption rate; mean+/−s.e.m. of n=4-6 biological replicates are shown in FIGS. 4C-4D.

FIG. 5A-5D. Subcellular targeting of SNORCLs to generate oxygen in the cytosol or mitochondria. 5A. Overview of mitochondrial targeted SNORCL. 5B. Immunoblots of mitochondrial and cytosolic fractions expressing FLAG-NdCld or mito-FLAG-NdCld. 5C. Seahorse intact cell oxygen consumption rate measurements at 1% ambient oxygen with sequential additions of piericidin+antimycin (1 mM each) and sodium chlorite (0, 0.5 mM, 1 mM, or 5 mM) in Hela cells expressing FLAG-NdCld or mito-FLAG-NdCld, with or without NIS. 5D. Raw traces of the oxygen levels within two minutes upon sodium chlorite addition (black arrow) in the Seahorse experiments shown in FIG. 5c. OCR: oxygen consumption rate; mean+/−s.e.m. of n=4-6 biological replicates are shown in FIGS. 5c-5d.

FIG. 6A-6C. 6A. Seahorse intact cell oxygen consumption rate measurements at 21% ambient oxygen with addition sodium chlorite (0, 1 mM, or 5 mM) in HeLa cells expressing GFP+NIS or NdCld+NIS. 6B. Seahorse intact cell oxygen consumption rate measurements at 21% ambient oxygen with sequential additions of piericidin+antimycin (1 uM each) and sodium chlorite (0, 1 mM, or 5 mM) in Hela cells expressing GFP+NIS or NdCld+NIS. 6C. Cell counts by Hoechst 33432 staining immediately after the Seahorse experiments, performed approximately 1 hour after chlorite addition. Means=standard deviations of n=4 samples are shown.

FIG. 7. HeLa cells expressing GFP (top left panel), NdCld (top right panel), GFP+NIS (bottom left panel), or NdCld+NIS (bottom right panel) were treated with freshly prepared sodium chlorite for 30 minutes, washed, and measured viability four hours later, Cell counts and viability were assessed after 3 days of growth using a Vi-Cell BLU Cell Viability Analyzer. Shown is the mean+/−s.d. of triplicate measurements.

FIGS. 8A-8D. 8A. Immunoblot analysis of FLAG-NdCld in Hela cells co-expressing NIS or mCherry. 8B. Seahorse permeabilized cell oxygen levels at 1% ambient oxygen with addition of sodium chlorite (0, 0.5 mM, 1 mM, or 5 mM) in HeLa cells expressing FLAG-NdCld+NIS or FLAG-NdCld+mCherry. 8C. Seahorse intact cell oxygen consumption rate measurements at 1% ambient oxygen with sequential additions of piericidin+antimycin (1 uM each) and sodium chlorite (0, 0.5 mM, 1 mM, or 5 mM) in Hela cells expressing FLAG-NdCld+NIS or FLAG-NdCld+mCherry. 8D. Traces of the oxygen levels within two minutes upon sodium chlorite addition (black arrow) in the Seahorse experiments shown in FIG. 8c. OCR: oxygen consumption rate; mean+/−s.e.m. of n=4-6 biological replicates are shown in FIGS. 8b-8d.

### DETAILED DESCRIPTION

Blood oxygen levels are routinely monitored in clinical medicine, and when required, we have facile means of delivering supplemental oxygen through nasal cannula, face masks, mechanical ventilation, and even extracorporeal membrane oxygenation. In contrast, we have few ways of providing supplemental oxygen within cells. Cells and organisms of course can be grown in chambers in which the ambient oxygen is regulated with gas mixtures (Ast and Mootha 2018). However, the poor solubility of oxygen in biofluids, its continuous exchange with the atmosphere, and active consumption by mitochondrial respiration, make it challenging to quickly manipulate intracellular oxygen levels with high spatiotemporal precision. Ideally, we would have an easy-to-use, genetically encoded capable of delivering on-demand, localized oxygen production inside living cells.

Here we sought to develop such a tool by harnessing naturally occurring enzymes that generate di-oxygen. While genetic tools exist for generating reactive oxygen species such as singlet oxygen (Shu 2011), no genetic tools for use in living cells have been described that generate molecular oxygen in its more familiar and stable triplet state. Enzymatic formation of the O—O bond is extremely rare. The most well appreciated and studied example is the water-splitting oxygen evolving complex (OEC) of photosystem II, which is central to oxygenic photosynthesis. The OEC contains numerous co-factors including heme, bicarbonate, chlorophyll, quinones, and a unique manganese cluster (Nicholls and Ferguson 2013). Oxygen can also be produced from methane oxidizing bacteria (Ettwig 2010). Another enzyme, called chlorite O2-lyase, chlorite: O2 lyase, or chlorite dismutase (all abbreviated “Cld”), converts chlorite (CO₂⁻) to oxygen (O₂) and chloride (Cl⁻) (reviewed in Hofbaur 2014).

We chose to focus on the Cld family of oxidoreductases as a chassis for a simple-to-use oxygen generator given that its substrate is bioorthogonal to eukaryotic metabolism. We show that when expressed in human cells, Cld enzymes exhibit high activity, and that we can co-express plasma membrane transporters that promote uptake of sodium chlorite for its subsequent intracellular conversion to oxygen. In this way we are able to successfully deploy a genetic system for Supplemental Oxygen Released from ChLorite (“SNORCL”; also sometimes called Supplemental Oxygen via Reduction of ChLorite).

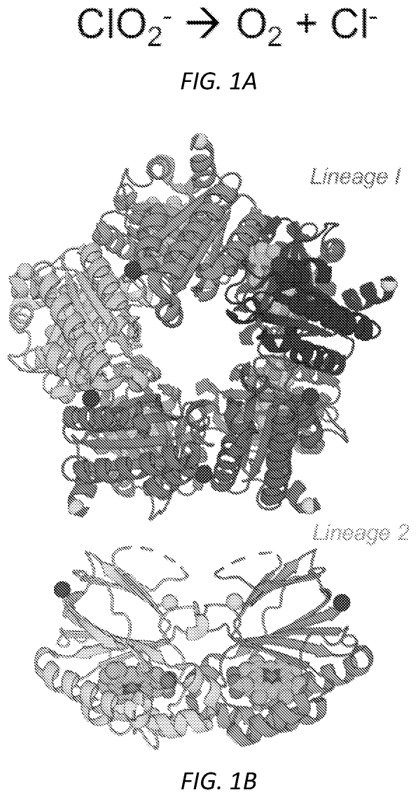

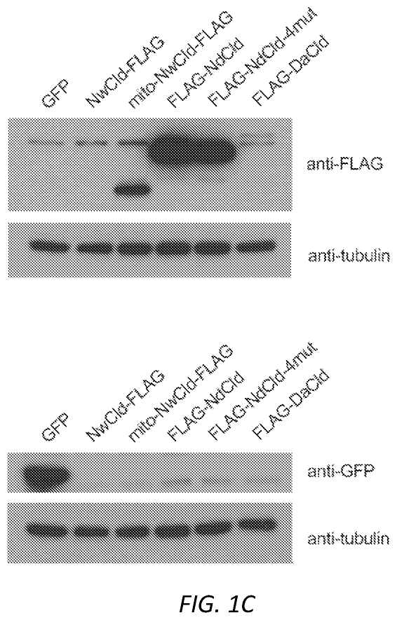

Cld oxidoreductases (EC 1.13.11.49) are distributed in bacteria and archaea and were originally discovered in 1996 in perchlorate respiring organisms (van Ginkel 1996). These enzymes catalyze the conversion of chlorite to oxygen and chloride (FIG. 1A). Cld enzymes are heme containing and can be homo-pentameric (Lineage I, found in Dechloromonas aromatica and Nitrospira defluvii) or homo-dimeric (Lineage II, found in Nitrobacter winogradskyi) (FIG. 1B). All characterized Cld enzymes possess an iron-containing heme b co-factor with histidine as the axial ligand, as well as a highly conserved arginine critical for catalysis (reviewed in Hofbaur 2014). Cld enzymes tend to be fast, do not generate reactive oxygen species, and can exhibit high turnovers before inactivating (Lee 2008). Although purified Cld enzymes have been proposed as in vitro enzymes for de-toxification or for studying rapid in vitro kinetics of oxygen dependent enzymes (Dassama 2012), to our knowledge, no prior studies have proposed to expressing them within eukaryotic cells for oxygen production.

Here we have introduced genetic SNORCLs for the facile generation of oxygen within living cells. Although oxygen is essential for all forms of life, including humans, at present, we have few or no means of being able to manipulate intracellular oxygenation inside cells or organisms with genetic control. The current state of the art for manipulating oxygen entails placing cultured cells or organisms in chambers in which the ambient oxygen can be controlled. Herein we have demonstrated that, optionally with the use of the NIS transporter, SNORCLs are able to generate intracellular oxygen lasting minutes to hours in cells that remain viable.

To our knowledge this is the first report of oxygen generation within mammalian or human cells.

### Cld Enzymes, Chlorite Transporters, and Expression Constructs

Described herein is the use of Cld enzymes, and optionally chlorite transporters, and expression thereof in eukaryotic cells.

Chlorite dismustases (Cld) are heme b-containing oxidoreductases that are found in bacteria including Proteobacteria, Cyanobacteria, and Nitrospirae, as well as in archaea. Cld useful in the present methods and compositions have chlorite decomposition activity; an exemplary Cld is homo-pentameric (Lineage I, e.g., from Dechloromonas aromatica (DaCld) and Nitrospira defluvii (NdCld)) or homo-dimeric (Lineage II, e.g., from Nitrobacter winogradskyi (NwCld)). See, e.g., Hofbauer et al., Biotechnol J. 2014 April; 9(4): 461-473; Kostan et al., J. Struct. Biol. 2010; 172:331-342; van Ginkel, Arch Microbiol. 1996 November; 166(5): 321-6; Goblirsch, B. et al. J Mol Biol 408(3): 379-98 (2011); Coates and Achenbach, Nat Rev Micro 2, 569-580 (2004) and U.S. Pat. No. 10,724,010. Exemplary sequences are known in the art and include those provided herein (optionally lacking the FLAG (DYKDDDDK (SEQ ID NO:1)) sequence and any linkers, e.g., GS-rich linkers (GGSGGSGGS (SEQ ID NO:2))) as well as those in the preceding references, particularly those disclosed in Table 1 of U.S. Pat. No. 10,724,010, including RefSeq accession numbers YP_005026408.1, YP_285781.1, AAM92878.1, WP_014235269.1, AAT07043.1, WP_009867516.1, CAC14884.1, WP_013516316.1, ACA21503.1, YP_004267835.1, EFH80711.1, YP_004178041.1, YP 004367213.1, YP_004058724.1, or YP_004172359.1. In preferred embodiments, the sequences useful herein have an arginine residue at the distal side of heme b (Hofbauer et al., Biotechnol J. 2014 April; 9(4): 461-473) required for chlorite degradation. The sequences should lack a periplasmic targeting sequence, and are preferably codon optimized for expression in a host cell. In some embodiments, the sequences include a signal targeting them to a specific subcellular compartment, e.g., a mitochondrial targeting presequence and/or internal signal, see, e.g., Truscott et al., Current Biology, Vol. 13, R326-R337, Apr. 15, 2003.

An exemplary sequence of NdCld lacking a periplasmic targeting sequence is: MADREKLLTESGVYGTFATFQMDHDWWDLPGESRVISVAEVKGLVEQWSGKILVESYLLRGL SDHADLMFRVHARTLSDTQQFLSAFMGTRLGRHLTSGGLLHGVSKKPTYVAGFPESMKTELQ VNGESGSRPYAIVIPIKKDAEWWALDQEARTALMQEHTQAALPYLKTVKRKLYHSTGLDDVD FITYFETERLEDFHNLVRALQQVKEFRHNRRFGHPTLLGTMSPLDEILEKFAQ (SEQ ID NO: 3). A useful sequence to target any of the Cld proteins described herein to the mitochondria comprises: MLATRVFSLVGKRAISTSVCVRAH (SEQ ID NO:4).

Transporters that promote uptake of chlorite include human sodium iodide symporter (NIS), encoded by SLC5A5, and homologs thereof, e.g., as shown in Table 1.

TABLE 1
Chlorite transporters

| | | |
|---|---|---|
| SLC5A5 | H. sapiens | NP_000444.1 |
| SLC5A5 | P. troglodytes | XP_524154.2 |
| SLC5A5 | C. lupus | XP_541946.3 |
| SLC5A5 | B. Taurus | XP_002688618.2 |
| Slc5a5 | M. musculus | NP_444478.2 |
| Slc5a5 | R. norvegicus | NP_443215.2 |
| SLC5A5 | G. gallus | XP_429095.4 |
| slc5a5 | X. tropicalis | NP_001116937.1 |
| slc5a5 | D. rerio | NP_001082860.1 |

Nucleic acid molecules that encode a Cld or chlorite transporter polypeptide as described herein encode a functional protein; a functional Cld has chlorite decomposition activity, and a functional transporter imports chlorite into a cell. The nucleic acid molecules can include a nucleotide sequence shown herein. In one embodiment, the nucleic acid molecule includes sequences encoding the human chlorite transporter protein (i.e., “the coding region” or “open reading frame”), as well as 5′ untranslated sequences. Alternatively, the nucleic acid molecule can include only the coding region, e.g., without any flanking sequences that normally accompany the subject sequence.

In some embodiments, a Cld or chlorite transporter includes a protein sequence that is at least about 85% or more homologous to the entire length of a sequence as shown herein. In some embodiments, the sequence is at least about 90%, 91%, 92%, 93%, 94%, 95%, 96%, 97%, 98%, 99% or 100% identical over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200, or more amino acids) in length.

TABLE 2
Cld enzymes

| | | |
|---|---|---|
| NdCld | Nitrospira defluvii | ACE75544.1 |
| DaCld | Dechloromonas | Q47CX0.1 |
| | aromatica | |
| NwCld | Nitrobacter winogradskyi | WP_011315650.1 |

Methods of alignment of sequences for comparison are well-known in the art. For example, the determination of percent sequence identity between any two sequences can be accomplished using a mathematical algorithm. Non-limiting examples of such mathematical algorithms are the algorithm of Myers and Miller (1988) CABIOS 4:11 17; the local homology algorithm of Smith and Waterman (1981) J. Mol. Biol. 147:195-7; the homology alignment algorithm of Needleman and Wunsch (1970) J. Mol. Biol. 48:443 453; the search-for-similarity-method of Pearson and Lipman (1988) Proc. Natl. Acad. Sci. 85:2444 2448; the algorithm of Karlin and Altschul (1990) Proc. Natl. Acad. Sci. U.S. Pat. No. 872,264, modified as in Karlin and Altschul (1993) Proc. Natl. Acad. Sci. USA 90:5873 5877.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn (aligning nucleotide sequences), the default parameters are Gap opening penalty=5 and Gap extension penalty=2. For blastp (aligning protein sequences), the default parameters are Gap opening penalty=11 and Gap extension penalty=1. For BLASTP, the defaults are wordlength of 3, and expectation (E) of 10, and the BLOSUM62 scoring matrix [see Henikoff and Henikoff, (1992) Proc Natl Acad Sci USA 89(22): 10915-10919] alignments (B) of 50, expectation (E) of 10, M=5, N=−4, and a comparison of both strand

In some embodiments, a nucleic acid sequence that encodes a Cld or chlorite transporter is used that has been codon optimized for expression in the cell, e.g., human codon optimized for expression in human cells. Nucleic acids encoding the Cld enzyme and/or the transporter can include mRNA or cDNA encoding the proteins, and the nucleic acids can be naked or in an expression vector, e.g., comprising a sequence such as a promoter that drives expression of the protein. The sequence can, for example, be in an expression construct.

In some embodiments, provided herein are nucleic acids comprising a fusion protein that is cleaved into separate the Cld and the transporter components following their expression as a single polypeptide (e.g., with the components separated by a protease cleavage site, a ribosomal skip sequence, or a 2A self-cleaving peptide sequence).

The fusion proteins can include one or more ‘self-cleaving’ 2A peptides between the coding sequences. 2A peptides are 18-22 amino-acid-long viral peptides that mediate cleavage of polypeptides during translation in eukaryotic cells. 2A peptides include F2A (foot-and-mouth disease virus), E2A (equine rhinitis A virus), P2A (porcine teschovirus-1 2A), and T2A (thosea asigna virus 2A), and generally comprise the sequence GDVEXNPGP (SEQ ID NO:5) at the C-terminus. See, e.g., Liu et al., Sci Rep. (2017) 7:2193. The following table provides exemplary 2A sequences.

| | | SEQ ID | |
|---|---|---|---|
| 2A | Coding Sequence | NO: | Source |
| F2A: | GCGCCAGTAAAGCAGACATTAAACTTT | 6 | STEMCCA |
| | GATTTCTGAAACTTGCAGGTGATGTAG | | (PMID: |
| | AGTCAAATCCAGGTCCA | | 20715179; |
| | | | Somers, et al. |
| | | | Stem Cells. 2010 |
| | | | October; 28(10): |
| | | | 1728-40) |
| F2A: | GGCAGCGGAAAACAGCTGTTGAATTTTG | 7 | pEB-C5 (PMID: |
| | ACCTTCTCAAGTTGGCGGGAGACGTGGA | | 25772473; Kim, et |
| | GTCCAACCCAGGGCCC | | al. Stem Cell |
| | | | Reports. 2015 Apr. |
| | | | 14; 4(4): 727-43) |
| P2A: | GCCACTAACTTCTCCCTGTTGAAACAAG | 8 | STEMCCA |
| | CAGGGGATGTCGAAGAGAATCCCGGGCCA | | (PMID: |
| | | | 20715179; |
| | | | Somers, et al. |
| | | | Stem Cells. 2010 |
| | | | October; 28(10): |
| | | | 1728-40) |
| E2A: | CAATGTACTAACTACGCTTTGTTGAAAC | 9 | STEMCCA |
| | TCGCTGGCGATGTTGAAAGTAACCCCGG | | (PMID: |
| | TCCT | | 20715179; |
| | | | Somers, et al. |
| | | | Stem cells. 2010 |
| | | | October; 28(10): |
| | | | 1728-40) |
| T2A: | GGCGGCGGGTCCGGAGGAGAGGGCAGAG | 10 | pEB-C5 (PMID: |
| | GAAGTCTTCTAACATGCGGTGACGTGGA | | 25772473; Kim, et |
| | GGAGAATCCTGGCCCA | | al. Stem Cell |
| | | | Reports. 2015 Apr. |
| | | | 14; 4(4): |
| | | | 727-43) |

Alternatively or in addition, the fusion proteins can include one or more protease-cleavable peptide linkers between the coding sequences. A number of protease-sensitive linkers are known in the art, e.g., comprising furin cleavage sites RX (R/K)R, RKRR (SEQ ID NO:11) or RR; VSQTSKLTRAETVFPDVD (SEQ ID NO:12); EDVVCCSMSY (SEQ ID NO:13); RVLAEA (SEQ ID NO:14); GGGGSSPLGLWAGGGGS (SEQ ID NO:15); TRHRQPRGWEQL (SEQ ID NO: 16); MMP 1/9 cleavage sequence PLGLWA (SEQ ID NO:17); TEV Protease sensitive linkers comprising ENLYFQ (G/S) (SEQ ID NO:18); Factor Xa sensitive linkers comprising I (E/D) GR; or LSGRDNH (SEQ ID NO:19) which is cleaved by cancer-associated proteases matriptase, legumain, and uPA. See, e.g., Chen et al., Adv Drug Deliv Rev. 2013 Oct. 15; 65(10): 1357-1369.

Calculations of identity between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

### Recombinant Expression Vectors

Also provided herein are vectors, preferably expression vectors, containing a nucleic acid encoding a Cld and/or chlorite transporter polypeptide as described herein, and optionally a nucleic acid encoding a chlorite transporter as described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a Cld or chlorite transporter nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce a Cld or chlorite transporter proteins.

The recombinant expression vector can be designed for expression of the Cld and chlorite transporter proteins in any eukaryotic cells. For example, Cld and chlorite transporter polypeptides can be expressed in animal cells, e.g., mammalian cells, e.g., human or non-human primate cells, rodent (e.g., rat, mouse, or hamster, e.g. CHO or COS cells), rabbit, cat, dog, cow, horse, goat, or other non-human mammals, or insect cells (e.g., using baculovirus expression vectors); or in fungus, e.g., in yeast cells. Thus the expression vector can be, e.g., a yeast expression vector, a vector for expression in insect cells, e.g., a baculovirus expression vector, or a vector suitable for expression in mammalian cells. When used in mammalian cells, the expression vector's control functions are often provided by viral regulatory elements. For example, commonly used promoters are derived from polyoma, Adenovirus 2, cytomegalovirus and Simian Virus 40.

### Genetically Engineered Cells and Organisms

The present methods and compositions can be used in any eukaryotic cells or non-human eukaryotic organisms, which are engineered to comprise a nucleic acid encoding a Cld as described herein and express a Cld enzyme from the nucleic acid, and optionally comprise a nucleic acid encoding a chlorite transporter as described herein and optionally express a chlorite transporter enzyme from the nucleic acid.

Thus provided herein are host cells that have been engineered to express a Cld and optionally a chlorite transporter nucleic acid molecule as described herein, optionally expressed from a recombinant expression vector or from sequences homologously recombined into the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The cells can be, for example, animal cells, e.g., mammalian cells, e.g., human or non-human primate cells, rodent (e.g., rat, mouse, or hamster, e.g. CHO or COS cells), rabbit, cat, dog, cow, horse, goat, or other non-human mammals, or insect cells (e.g., using baculovirus expression vectors); or fungus, e.g., yeast cells. In some embodiments, the cells are immortalized cells that can be kept in culture. Other suitable host cells are known to those skilled in the art, see, e.g., Goeddel, (1990) Gene Expression Technology: Methods in Enzymology 185, Academic Press, San Diego, CA. In some embodiments, the cells are human CAR-T cells, i.e., T cells that express chimeric antigen receptors (CARs) (Aghajanian et al., Nature Metabolism 4:163-169(2022); Gumber and Wang. EBioMedicine. 2022 March; 77:103941; Sterner and Sterner, Blood Cancer J. 2021 Apr. 6; 11(4):69. Preferably, the host cells do not express an endogenous chlorite transporter. In some embodiments, the host cells are not Saccharomyces cerevisiae, Saccharomyces monacensis, Saccharomyces bayanus, Saccharomyces pastorianus, Saccharomyces carlsbergensis, Saccharomyces pombe, Trichoderma reesei, Neurospora crassa, Kluyveromyces marxiamus, Kluyveromyces lactis, Kluyveromyces fragilis, Pichia stipitis, Pichia pastoris, Sporotrichum thermophile, Candida shehatae, Candida tropicalis, Neurospora crassa, Zymomonas mobilis, Clostridium saccharoperbutylacetonicum, Clostridium phytofermentans, Clostridium thermocellum, Clostridium beijerinckii, Clostridium acetobutylicum, Clostridium botulinum, Clostridium butyricum, Clostridium diolis, Clostridium ljungdahlii, Clostridium aerotolerans, Clostridium cellulolyticum, Clostridium tyrobutyricum, Clostridium pasteurianum, Moorella thermoacetica, Escherichia coli, Klebsiella oxytoca, Thermoanaerobacterium saccharolyticum, Yarrowia lipolytica, or Bacillus subtilis.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

Also provided are uni- and multicellular transgenic eukaryotic organisms comprising at least one cell that expresses Cld and optionally a chlorite transporter. In some embodiments, every cell in the organism expresses Cld and optionally a chlorite transporter. The organism in some embodiments of these aspects may be an animal; for example a non-human mammal such as a mouse. The organism may be an arthropod, e.g., an insect such as a fruit fly, or a worm such as Caenorhabditis elegans. The organism also may be a plant or protist, e.g., algae. Further, the organism may be a fungus, e.g., yeast. Methods for generating transgnic organisms are known in the art.

### Methods of Use

The present methods can include maintaining the cells and organisms described herein in an environment that includes chlorite, e.g., levels of chlorite about the normal environment for the cells or organisms. For example, for eukaryotic cells, e.g., in culture, the methods can include culturing the cells in a media comprising added chlorite, e.g., 50 μm to 5 mM chlorite, preferably at least 70, 75, 100, 250, or 500 UM chlorite, up to 1, 2.5 or 5 mM chlorite. For transgenic non-human uni- or multi-cellular eukaryotic organism, the methods can include maintaining the organisms an environment comprising chlorite, e.g., an aqueous environment comprising chlorite, or a gaseous environment comprising chlorite, e.g., sodium hydrogen chlorite (NaHClO₂). The chlorite can be, e.g., sodium chlorite (NaClO₂), chlorous acid (HClO₂), or a heavy metal chlorite (Ag+, Hg+, Tl+, Pb2+, Cu2+ or NH+₄).

The present methods (e.g., SNORCL) can be used as a genetic tool in research settings to acutely evolve oxygen on demand in cultured cells or in model organisms. For example, SNORCL can be targeted to different subcellular compartments for localized oxygen production. Such studies can provide insight into the biology of anoxia, as well as the toxicity of hyperoxia (Ast and Mootha 2019). SNORCLs could serve as genetic tools for studies of “causal metabolism,” specifically to evaluate the causal role of oxygen in processes or diseases of interest.

Beyond the research arena, the SNORCL technology could have many medical and biotechnological applications. For example, it could be delivered as a gene therapy to target tissues and alleviate hypoxia-mediated diseases. Alternatively, SNORCL may be useful in boosting the activity of cellular therapies such as CAR-T, where hypoxia in the tumor microenvironment contributes to T cell exhaustion (Schurich 2019). Organisms genetically modified to express SNORCL may even promote survival in extra-terrestrial, anoxic zones where chlorite is present (Mustard 2008; Hecht 2009).

### Examples

The invention is further described in the following examples, which do not limit the scope of the invention described in the claims.

### Materials and Methods

The following materials and methods were used in the Examples set forth below.

### Sequences

GFP was obtained from Addgene #19319, pLJM1-eGFP. mCherry was from Addgene #32383, pcDNA3.1-Peredox-mCherry. All other sequences were custom designed and synthesized for use in this study.

### Generation of Cell Lines Stably Expressing Transgenes

Cld enzymes and sodium/iodide symporters were stably expressed in HeLa cells using lentiviral transduction. Briefly, gene constructs were custom synthesized in pUC57-Kan (GenScript) with NheI and EcoRI restriction sites at the 5′ and 3′ ends, respectively. Cld cDNA was subcloned into the pLYS1 lentiviral expression vector (Addgene #50057), while SLC5A5 cDNA was subcloned into pLYS5 (Addgene #50054). Construct sequences were verified by Sanger sequencing (Azenta). Lentivirus was generated in 293T cells (ATCC #CRL-3216). 10⁶ cells were seeded per dish in 6 cm culture dishes, in 5 ml media. The next day, the cells were transfected using X-tremeGENE HP transfection reagent (Roche #6366244001) with 1 μg of lentiviral construct, along with 900 ng psPAX2 (Addgene #12260) and 100 ng pCMV-VSV-G (Addgene #8454) lentiviral packaging and envelope plasmids. After forty-eight hours, lentivirus was collected and passed through a 0.45 um polyethersulfone syringe filter (Whatman #6780-2504). For lentiviral transduction, 2×10⁵ HeLa cells (ATCC #CCL-2) The next day, cells were treated with 8 μg/ml polybrene (Sigma #H9268) and transduced with 400 ul lentivirus. After 48 hours, cells were passaged and selected with 2 μg/ml puromycin (Gibco #A1113803) or 100 μg/ml hygromycin B (Sigma #H3274), as appropriate. Once fully selected, cells were maintained in puromycin or hygromycin B for an additional passage prior to use for subsequent experiments. HeLa cells were maintained in Dulbecco's Modified Eagle Medium (DMEM, Gibco #11995-065) supplemented with 10% fetal bovine serum (FBS, Sigma #2442), 1× GlutaMax (Gibco #35050061), and penicillin/streptomycin (Gibco #15140122). Cells were maintained in a 37° C., 5% CO2 incubator.

### Immunoblot Analysis

For Western Blots from HeLa cell lysates, cells were first washed with ice cold PBS, then lysed with ice cold 1% Triton lysis buffer ( ) supplemented with protease/phosphatase inhibitor (Cell Signaling #5872). Lysates were clarified by centrifugation at 21,000×g for 10 min, at 4 C. Supernatants were transferred to clean microcentrifuge tubes on ice. Protein content was quantified by Bradford assay (Bio-Rad #5000205). Samples were normalized to 1 ug/ul in lysis buffer with 1×SDS sample buffer (2% SDS, 5% β-mercaptoethanol, 5% glycerol, 47.4 mM Tris HCl, 16.6 uM Bromophenol Blue, pH 6.8). Samples were heated for 5 min at 95C on a heat block, and cooled at room temperature before loading on SDS-PAGE gels. Samples were run on Tris-Glycine gels at 120 volts for approximately 2 hours, then transferred to PVDF membranes (Bio-Rad #1704157) using a Trans-Blot Turbo Transfer System (Bio-Rad). Membranes were blocked in 5% milk/TBST for 1 hour at room temperature. Membranes were probed with anti GFP (Abcam #ab6556), anti-FLAG (Cell Signaling #2368), or anti-β-tubulin (Cell Signaling #2128) diluted 1:1000 in 5% milk/TBST, incubated overnight at 4 C. HRP-conjugated donkey anti-rabbit (Cell Signaling #7074) secondary antibody was used at 1:10,000 dilution in 5% milk/TBST for 1 hour at room temperature. Membranes were washed 6×5 minutes with 1×TBST before and after secondary antibody incubation. Membranes were incubated with Western Lightning Plus ECL substrate (PerkinElmer #NEL104001EA) for 3 minutes. Luminescence was detected using Amersham Hyperfilm ECL film (GE Healthcare #28906838) developed on an X-Omat 2000A Processor (Kodak).

### Purification and Biochemical Characterization of NdCld Expressed in Human Cells

HeLa cells were harvested, washed in PBS, and resuspended in buffer A containing 300 mM NaCl, 50 mM HEPES pH7.4, 2% glycerol, complete EDTA-free protease inhibitor cocktail (Roche), PMSF, and Benzoase (Millipore Sigma). Cells were lysed with 10 strokes of a tight Dounce homogenizer followed by a total of 90 seconds of sonication on ice. The suspension was centrifuged at 25,000×g for 1 hour and the resulting lysate was incubated with anti-FLAG M2 affinity gel (Millipore Sigma) for 90 minutes. The slurry was loaded into a gravity flow column, the flow through collected, and the resin washed with 20 column volumes of buffer A (without the protease inhibitors and nuclease). The protein was eluted using multiple incubations of the resin in buffer A containing 100 ug/ml 3×FLAG peptide. The collected protein was concentrated via Amicon 10KD centrifugal filters (Millipore Sigma), filtered, and then loaded onto a Superdex 200 Increase 5/150 GL gel filtration column (Cytiva) equilibrated with 100 mM NaCl, 20 mM HEPES pH 7.4, and 0.2% glycerol. Sizing of the protein through gel filtration was accomplished by comparison to a gel filtration standard (Bio-Rad) run under identical buffer, flow rate, and temperature conditions.

### Assessment of Heme Content in Purified Protein

Heme incorporation was measured through the pyridine hemochromagen assay (Barr and Guo, 2015). Spectra were collected using a Nanodrop One C. Equal volumes of purified NdCLD (9.4 uM) and a solution of 0.2 M NaOH, 40% (v/v) pyridine, and 500 uM potassium ferricyanide were mixed to generate the oxidized spectra. Sodium dithionite was then added to a final concentration of 2.5 mM in order to obtain the reduced spectra. The heme concentration was then determined from the absorbance at 557 nm of the reduced NdCLD sample using the heme extinction coefficient 34.7 mM-1 cm-1 (Paul et al, 1953). The calculated heme concentration, 4.6 uM, corresponded to 98% incorporation of heme in the purified NdCLD.

### Steady State Kinetics of NdCld in Permeabilized Human Cells

HeLa cells were pelleted at 800×g for 3 min, washed with PBS, pelleted again, and then resuspended in assay buffer (125 mM KCl, 2 mM K2HPO4, 1 mM MgCl₂, 2 0 mM HEPES pH 7.2, 5 mM glutamate, 5 mM malate, and 0.01% digitonin) at a concentration of 5×10⁶ cells/ml. Oxygen production was measured using a FireSting optical oxygen meter connected to a sensor vial. One ml of cell solution (5×10⁶ cells) was used for each measurement. Measurements were performed under ambient air conditions with stirring of the cell solution. The reaction was initiated by adding sodium chlorite solution (prepared in assay buffer) to predetermined concentrations. The initial rates were determined from the resulting oxygen traces using up to 20 seconds of the linear portion of the trace via the ICEKAT web server (Olp, 2020). The means of 3 replicate rates were plotted against the chlorite concentrations to estimate the K_{M}.

### Three-Day Toxicity of Sodium Chlorite in Human Cells

HeLa cells cells were trypsinized, counted, and prepared at 10⁵ cells/ml in normal growth media. 1 M sodium chlorite stock solution was prepared fresh at the time of the assay in UltraPure dH2O, and diluted to 2× working concentrations in cell growth media. Cells were seeded in 24-well plates, with triplicate wells for each condition. 500 ul of each 2× chlorite/media preparation was first added to the plate. 500 ul of cell suspension (5×10⁴ cells) was then added to each well. The plate was gently mixed, and cells were grown for 3 days in a 37° C./5% CO2 incubator. After 3 days, cells were washed briefly with 500 ul of PBS, trypsinized with 250 ul TrypLE Express, and resuspended with 750 ul of normal growth media to 1 ml total volume. In wells containing a majority of visibly dead, floating cells, cells were resuspended by vigorously pipetting up and down rather than by trypsinization. 200 ul of each cell suspension was then quantified using a Vi-Cell BLU Cell Viability Analyzer (Beckman Coulter).

### Measurement of Oxygen and Oxygen Consumption Rates in Intact Cells

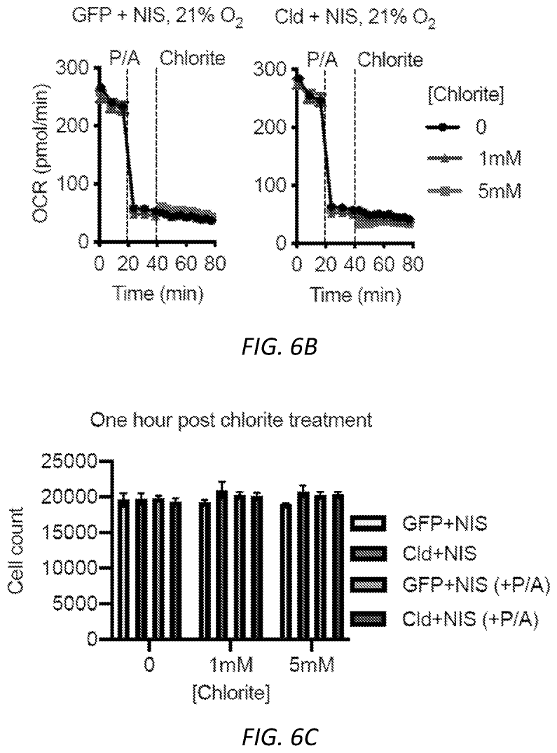

For OCR and O₂ measurements in HeLa cells using the Agilent Seahorse XFe96 system, cells were seeded at 1.5×10⁴ cells in 80 ul/well in 96-well Seahorse cell culture plates, in DMEM (Gibco #11995-065) supplemented with 10% FBS (Gibco #26140-079) and penicillin/streptomycin (Gibco #15140-122). After 16-20 hours, 175 ml of HEPES buffered Seahorse DMEM supplemented with 10 mM glucose, 1 mM sodium pyruvate, and 2 mM L-glutamine (Agilent) was added, and the plate was transferred to a 37° C. non-CO₂ incubator for one hour. The Seahorse cartridge was hydrated according to the manufacturer's protocol. Piericidin A (Enzo Life Sciences)+Antimycin A (Sigma) and sodium chlorite (Sigma) were prepared in Seahorse DMEM and added to the wells by injections during the Seahorse run. Three or four baseline respiratory rate measurements were taken, followed by sequential injections of Piericidin A+Antimycin A (three or four measurements) and sodium chlorite (12 measurements). To confirm uniform cell numbers across cell lines and no striking changes in cell numbers over the course of a Seahorse experiment (FIG. 6C), after the Seahorse run 2 mg/mL Hoechst 33342 (Invitrogen) was added to each well and incubated for 10 min, and the plate was imaged on a BioTek Cytation 5 Cell Imaging Multi-Mode Reader. The total number of nuclei (a proxy for the cell number) in each well was determined. To perform Seahorse measurement at 1% ambient oxygen, a XFe96 system was set up in a Coy O2 Control In Vitro Glove Box. Hydrated Seahorse cartridge, Seahorse DMEM, and other reagents were incubated at 1% ambient oxygen in the glove box overnight prior to the Seahorse experiment. During the Seahorse run at 1% ambient oxygen, the “Hypoxia mode” was used according to Agilent's protocol. Freshly prepared sodium sulfite solution was loaded into the cartridge to provide a “zero” oxygen reference.

For permeabilized Seahorse OCR measurements, HeLa cells were seeded at 1.5×10⁴ cells/well in 80 ul/well growth media and grown overnight at 37° C. Seahorse cartridges were hydrated overnight at 37° C., according to the manufacturer's protocol. After 16-20 hours, cells were washed once with MAS buffer (70 mM sucrose, 220 mM mannitol, 5 mM KH₂PO₄, 5 mM MgCl₂, 2 mM HEPES, 1 mM EGTA, 0.2% FA-free BSA). Cells were then permeabilized with MAS buffer supplemented with 2 nM XF Plasma Membrane Permeabilizer (Agilent 102504-100) and 1 uM each of Piericidin A+Antimycin A. Upon assay start, six baseline respiratory rate measurements were taken, followed by injection of chlorite and twelve respiratory rate measurements after chlorite injection. Permeabilized Seahorse experiments were also performed at 1% ambient oxygen, in a Coy O₂ Control In Vitro Glove Box as described above.

### Example 1. A Genetically Encoded System for Oxygen Generation Inside Living Human Cells

We began by testing the expression of several naturally occurring Cld variants as well as those engineered for greater thermostability or subcellular localization (Netzer 2018). To facilitate expression and purification from human HeLa cells, Cld genes were engineered through codon optimization, deletion of predicted periplasmic targeting sequences, and incorporation of epitope tags at the termini least likely to impact enzyme activity as suggested by published pentameric and dimeric CLD structures. We tested enzymes from both lineage 1 (FLAG-NdCld, FLAG-DaCld) and lineage 2 (NwCld-FLAG), including one targeted to mitochondria (mito-NwCld-FLAG). We also used computational methods (16) to design four point mutations predicted to improve NdCld thermostability (FLAG-NdCld^{4xMUT}). In these preliminary screens we saw the greatest expression from N-terminally FLAG-tagged NdCld (FIG. 1C), which became the focus of our study. Cells expressing FLAG-NdCld appeared healthy, comparable to cells expressing GFP, without any obvious impact on cell morphology or growth.

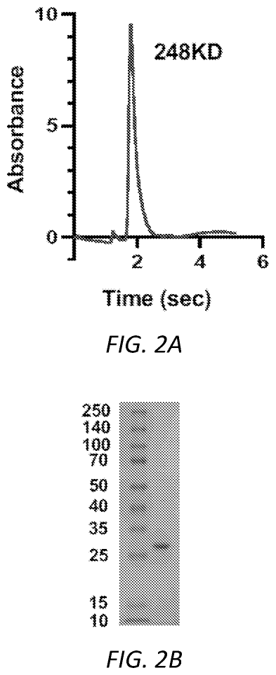

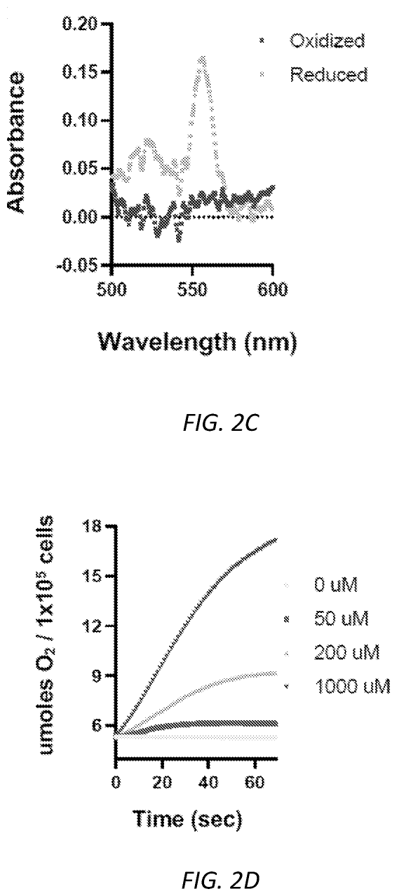

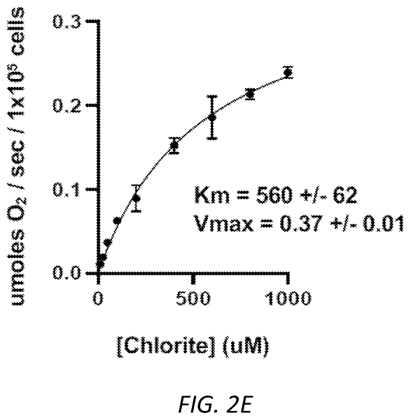

We next sought to determine whether FLAG-NdCld expressed in human cells grown in ambient conditions at sea level was properly assembled with its heme b co-factor. We cultured cells expressing FLAG-NdCld and performed affinity purification under non-denaturing conditions. The purified enzyme is monodispersed, as shown by gel filtration chromatography, running at an apparent molecular weight of 248 kDa (FIG. 2A). SDS-PAGE analysis results in a clean, Coomassie-stained band at the expected molecular weight of 29 kDa (FIG. 2B). Given that Nd-Cld, as well as other Lineage I Clds, are known to form pentamers (Kostan 2010), we speculate that in our mild detergent conditions, the enzyme is running as a dimer of pentamers. The oxidized and reduced spectra, obtained by addition of ferricyanide or dithionite, respectively, confirms that the enzyme expressed in human cells incorporates a heme b cofactor (FIG. 2C). We quantified the heme concentration from the absorbance at 557 nm of the reduced NdCld sample, and assuming a heme extinction coefficient of 34.7 mM⁻¹ cm⁻¹ (Paul 1953), we estimate 98% incorporation of heme in the purified NdCLD.

We next characterized the activity of this protein in human cell extracts. We permeabilized HeLa cells with digitonin and then performed a dose response experiment with addition of sodium chlorite. Doses spanning 10 uM to 1 mM were used, as previous studies have shown that higher chlorite concentrations lead to inactivation of the enzyme (Hofbauer 2014). We monitored oxygen evolution using an optical probe in a well-stirred, air saturated cuvette. We observe very fast and strong oxygen evolution in response to added sodium chlorite (FIG. 2D), consistent with what has been reported for bacterial expressed and purified enzyme. For the bacterial expressed NdCld, a broad range of Kₘ values have been reported, ranging from 58-69 uM for the purified enzyme (Kostan 2010; reviewed in Hofbauer 2014), to as high as 15.8 mM in the original characterization of NdCld in E. coli extracts (Maixner 2008). In our human digitonin-permeabilized cell extract, based on initial rates of oxygen evolution, we estimate that the Kₘ for chlorite is 560 uM and the Vmax is 0.37 umoles/second/100,000 cells (FIG. 2E). Our kinetic parameters for human HeLa cell extracts expressing NdCld are far superior to those reported in bacterial extracts, but our observed velocity is less than what has been reported for the the purified enzyme, likely because of active oxygen consumption by these extracts and presence of HEPES and chloride which have been shown to have a detrimental impact on Cld activity (Freire 2015; Streit 2008). Regardless, these studies demonstrate that NdCld enzymes can be safely expressed in human cells grown in standard cell culture conditions, they oligomerize, fully incorporate the heme b co-factor, and function in a highly robust manner with rapid production of oxygen. These studies demonstrate that NdCld enzymes can be safely expressed in human cells grown in standard cell culture conditions. They oligomerize, fully incorporate the heme b co-factor, and in permeabilized extracts, function properly with rapid production of oxygen from chlorite.

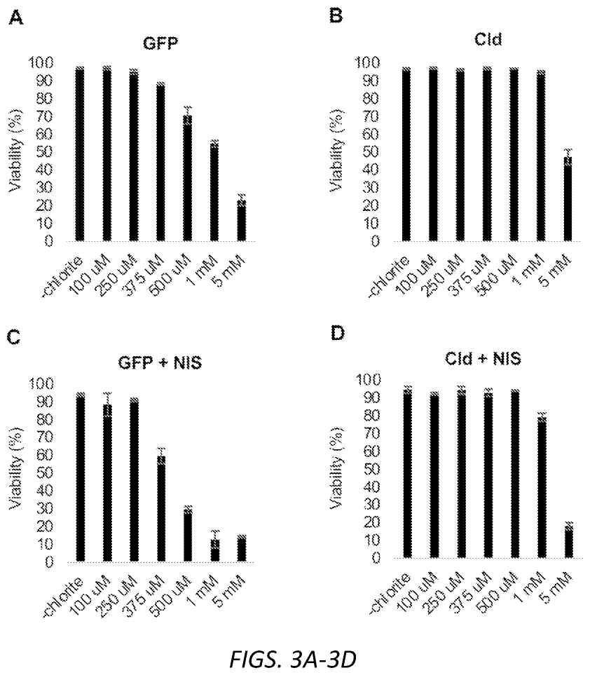

For NdCld to be useful in intact cells, sodium chlorite would have to transit through the plasma membrane at doses tolerated for the specific application. However, as chlorite is negatively charged and polar, it is not expected a priori to rapidly diffuse into cells across the plasma membrane. Nonetheless, previous studies have shown that at very high doses, chlorite compromises fitness and growth of cells due to its oxidant properties (Ali 2016). Chlorite is an oxidant, and at high doses, can damage human erythrocytes (Ali 2016). In yeast, a 4 mM dose is required to achieve 50% growth inhibition (Kwolek-Mirek 2011). In order to both verify that Cld was active in HeLa cells under normal growth conditions as well as confirm that the cells expressing NdCld were healthy in the presence of chlorite, we performed a three-day toxicity study of HeLa cells bathed in chlorite-containing growth media. In HeLa cells, we observed a 50% decrease in viability when cells were treated with ˜2 mM of sodium chloride for 3 days (FIG. 3A). However, the toxicity was alleviated by the expression of NdCld (FIG. 3B). These data suggest that at a high dose chlorite can enter HeLa cells over a three-day period, and that it is toxic in a way that can be alleviated by expression of NdCld.

Using this three day toxicity assay, we screened for transporters that might promote uptake of chlorite into human cells. To our knowledge, no study has ever investigated chlorite transport, though transport activity for the polyatomic anions nitrate, nitrite, and chlorate have been reported. We expressed both wild-type and activity boosting point mutants of nitrate transporters from A. thaliana, A. nidulans, H. polymorpha, and human, without any obvious boost in chlorite toxicity (data not shown). We then turned to the human sodium iodide symporter (NIS), encoded by SLC5A5 (Eskandari 1997). The human NIS is expressed as a homodimer on the basolateral membrane of thyroid follicular cells with a C-in, N-out topology, where it electrogenically concentrates iodide with symport of 2 Na⁺ ions. Electrophysiological studies of the NIS in Xenopus oocytes shows it has broad transport activity for many anions, including chlorate (ClO₃⁻) with a Kₘ of 277 uM (Eskandari 1997). When we expressed the human NIS in HeLa cells, we observed a five-fold increase in the three-day toxicity of added sodium chlorite (FIG. 3C) that could be attenuated by NdCld co-expression (FIG. 3D). Without being bound by theory, the most parsimonious explanation our results is that NIS promotes chlorite uptake into the HeLa cells, and NdCld catalyzes its conversion from chlorite to molecular oxygen and chloride.

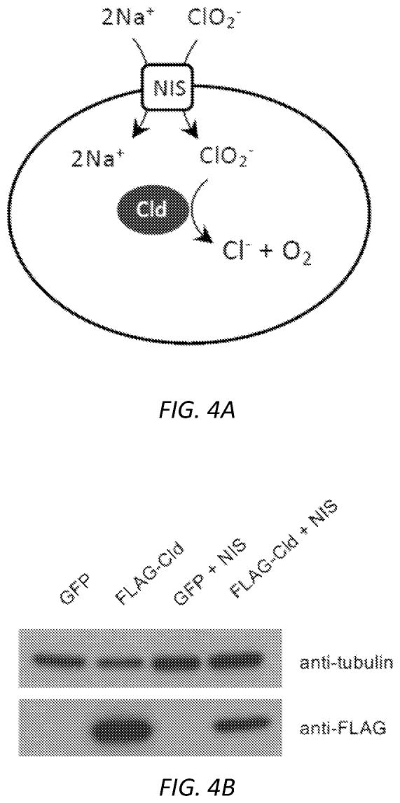

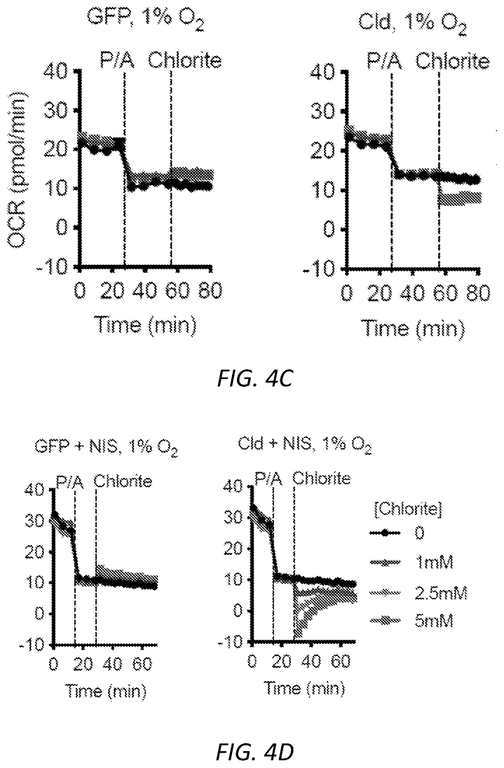

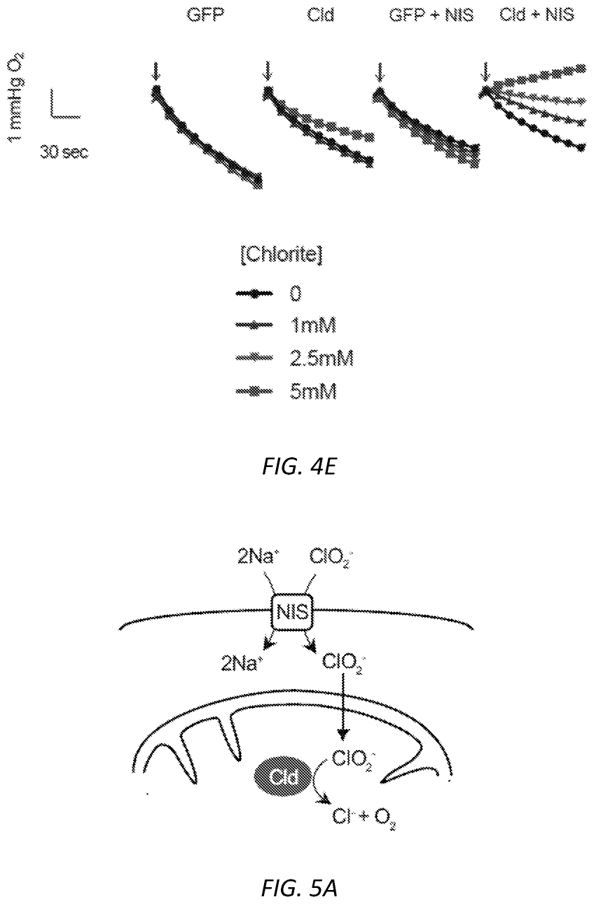

We also sought to determine whether we could detect oxygen evolution in intact cells using SNORCLs (FIG. 4A). We grew Hela cells expressing either FLAG-NdCld or GFP, with or without the co-expression of NIS (FIG. 4B), for measurements of oxygen consumption rate (OCR) using the Seahorse XFe96 Analyzer. We anticipated challenges in being able to detect oxygen evolution by SNORCLs given that any oxygen it generates could rapidly equilibrate with the atmosphere, and second, mitochondria could actively consume it. After initial experiments at 21% oxygen (FIGS. 6A-6B), where we did observe modest but reproducible oxygen generation in a Cld-dependent manner, we performed these experiments in a 1% ambient oxygen environment (to prevent back diffusion) while treating cells with piericidin and antimycin (to block mitochondrial respiration).

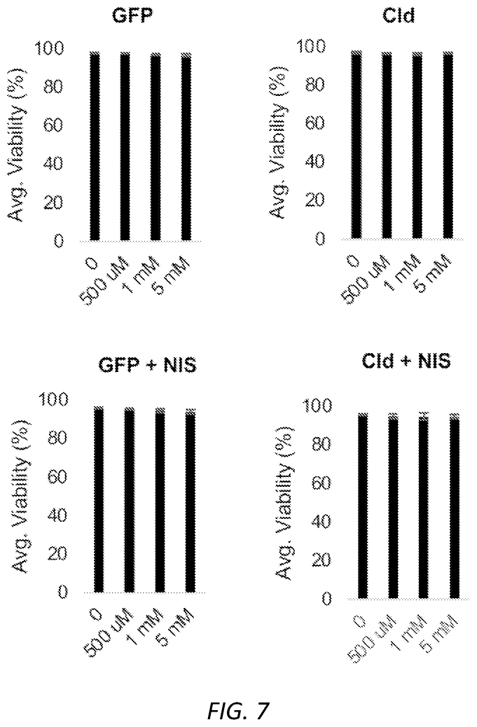

Under these conditions, oxygen generation, as evidenced by a decline in apparent OCR, was immediately obvious and striking in cells co-expressing both NdCld and NIS, where we saw robust oxygen production with with a clear dose response beginning with 1 mM chlorite (FIGS. 4C-4D). In these experiments maximal rates of oxygen evolution occurred during the first ten minutes, but then continued for more than a total of 30 minutes. Cells remained viable throughout the course of these Seahorse experiments even one hour after addition of the highest doses of chlorite (FIG. 6C). In separate experiments, found no dimunition in viability four hours following a 30 minute exposure to high does chlorite (FIG. 7). Examination of the oxygen partial pressures from the Seahorse traces (FIG. 4E) clearly shows a chlorite-dose dependent oxygen evolution in these intact cells in a way that is boosted with co-expression of NIS, which is clearly important given that Cld protein levels appear slightly lower in cells co-expressing NIS (FIG. 4B). Partial pressure of oxygen reported by the Seahorse instrument (FIG. 4E) clearly shows a chlorite dose-dependent oxygen evolution in these intact cells in a way that is boosted by co-expressing NIS (FIG. 4E). Collectively these studies provide definitive proof that SNORCLs permit on-demand oxygen generation within living human cells.

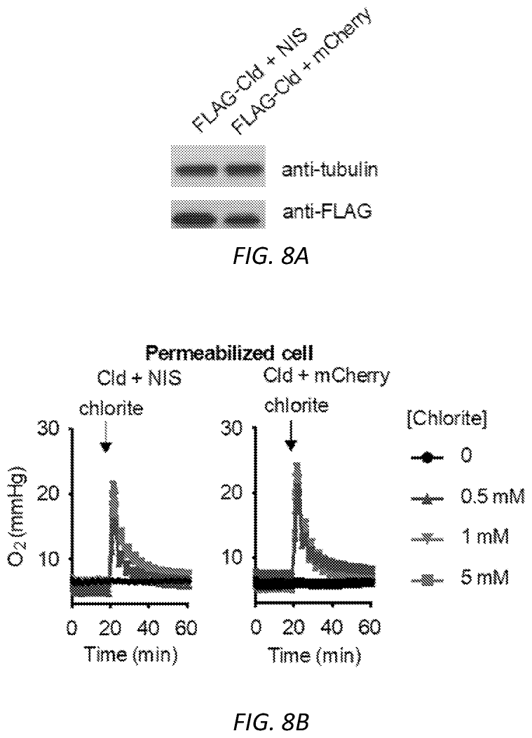

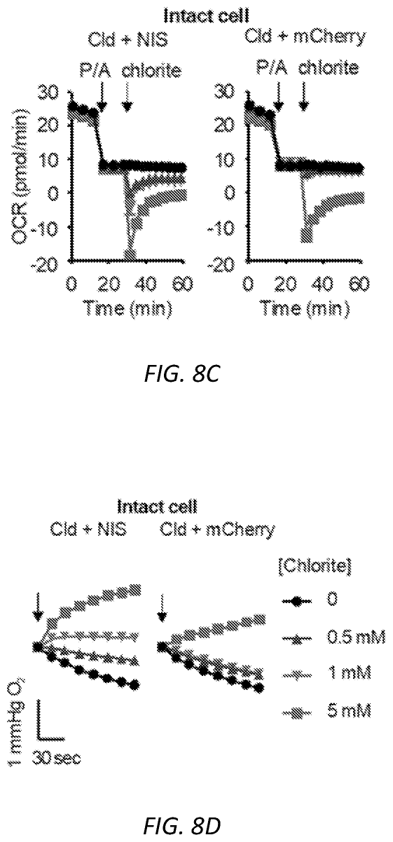

To further confirm that oxygen generation was taking place inside the cell, we performed an independent set of experiments in which we measured oxygen with both permeabilized and intact cells (FIGS. 8A-8D). In this set of experiments, we again expressed FLAG-NdCld, and this time co-expressed either NIS or mCherry, the latter serving as a viral transduction and antibiotic selection control. In both cell lines, we saw robust protein expression of FLAG-NdCld (FIG. 8A). When the plasma membrane is permeabilized, both cell lines exhibit comparable dose responses to injected sodium chlorite (FIG. 8B). In intact cells, although we were able to generate oxygen pulses in both cell lines at a high dose of 5 mM chlorite, we observed oxygen evolution even with 500 uM or 1 mM of chlorite in NIS but not mCherry expressing cells (FIGS. 8C-8D). These studies further confirm that co-expressing the NIS facilitates the transport of the chlorite into cells.

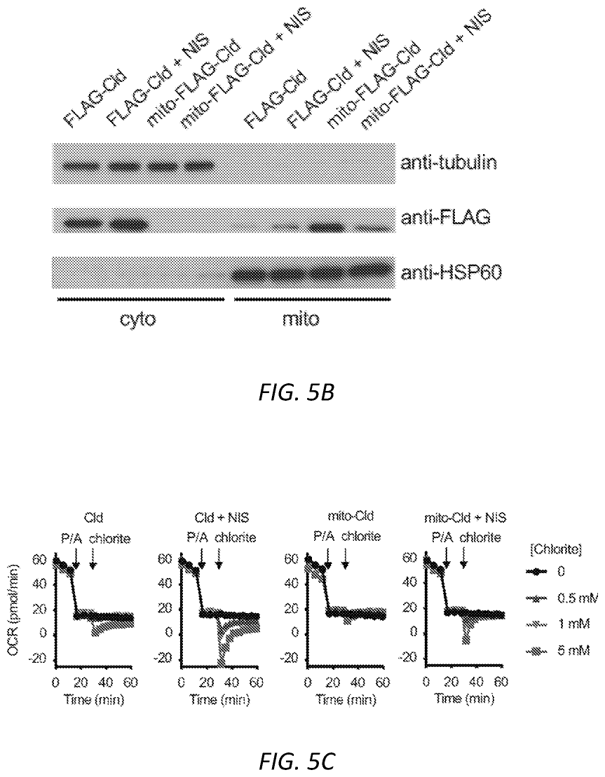

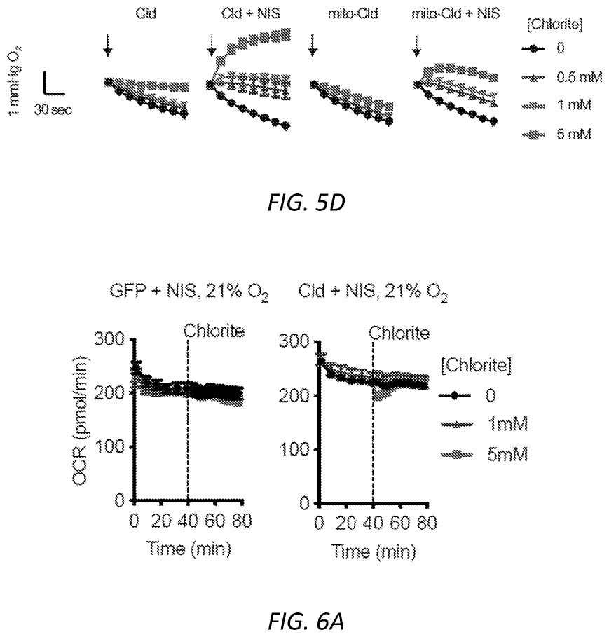

Finally, we sought to determine whether we could genetically target the SNORCL system to different subcellular compartments (FIG. 5A). We introduced an N-terminal mitochondrial targeting sequence to FLAG-NdCld (mito-FLAG-NdCld) and compared it to FLAG-NdCld. These constructs successfully targeted the enzyme to mitochondria and the cytosol, respectively, based on immunoblot analysis of respective cell fractions (FIG. 5B). To determine whether the mitochondrial targeted NdCld can function, we performed Seahorse analysis in intact cells, and found that the mito-FLAG-NdCld is also capable of generating oxygen in response to added chlorite (FIG. 5C). Examination of the oxygen partial pressures from the Seahorse instrument (FIG. 5D) confirms net generation of oxygen by mito-FLAG-NdCld when NIS is co-expressed. These experiments support that chlorite entering into the cell is able to be taken up by mitochondria, presumably via mitochondrial anion transporters. Collectively these studies provide proof that SNORCL can be targeted to mitochondria to permit on-demand oxygen generation with spatiotemporal resolution.

### Partial Listing of Sequences

Listed below are the human codon optimized DNA and corresponding protein sequences used in this study, which provide examples of sequences usable in the methods and compositions described herein (optionally omitting the FLAG (DYKDDDDK (SEQ ID NO:1)) sequence and any linkers, e.g., GS-rich linkers (GGSGGSGGS (SEQ ID NO:2))). All other sequences were custom designed and synthesized for use in this study.

FLAG-NdCld:
SEQ ID NO: 20)
GCTAGCATGGATTACAAGGATGACGATGACAAGGGTGGATCTGGT
GGATCTGGTGGATCTGCCGACCGGGAAAAGCTGCTGACCGAGAGC
GGTGTTTACGGCACATTCGCTACATTTCAGATGGACCATGATTGG
TGGGACCTGCCTGGCGAATCCAGAGTGATCAGCGTGGCTGAAGTG
AAGGGCCTGGTCGAGCAGTGGAGCGGAAAGATCCTGGTGGAATCT
TATCTGCTGAGAGGCCTGAGCGACCACGCCGATCTGATGTTCAGA
GTGCACGCCAGAACCCTGTCTGATACCCAGCAGTTCCTGAGCGCC
TTTATGGGCACCAGGCTGGGCAGACACCTGACCAGCGGAGGACTT
CTGCACGGCGTGTCCAAGAAACCTACATACGTGGCCGGCTTCCCC
GAGTCTATGAAAACAGAGCTGCAGGTCAACGGCGAGAGCGGCAGC
AGACCTTACGCCATCGTGATTCCTATCAAGAAGGACGCCGAATGG
TGGGCCCTGGACCAGGAGGCCAGAACAGCCCTGATGCAGGAGCAC
ACCCAGGCAGCTCTGCCATACCTGAAGACCGTGAAAAGAAAGCTG
TACCACAGCACCGGCCTGGACGACGTGGACTTCATCACCTACTTC
GAGACAGAGCGGCTGGAAGATTTTCACAACCTGGTGCGGGCCCTG
CAACAAGTGAAGGAGTTCAGACACAATCGGCGCTTCGGCCACCCT
ACCCTGCTGGGCACCATGAGCCCCCTGGATGAGATCCTCGAGAAG
TTCGCCCAGTGAGAATTC
SEQ ID NO: 21)
MDYKDDDDKGGSGGSGGSADREKLLTESGVYGTFATFQMDHDWWD
LPGESRVISVAEVKGLVEQWSGKILVESYLLRGLSDHADLMFRVH
ARTLSDTQQFLSAFMGTRLGRHLTSGGLLHGVSKKPTYVAGFPES
MKTELQVNGESGSRPYAIVIPIKKDAEWWALDQEARTALMQEHTQ
AALPYLKTVKRKLYHSTGLDDVDFITYFETERLEDFHNLVRALQQ
VKEFRHNRRFGHPTLLGTMSPLDEILEKFAQ*
FLAG-NdCld^{4MUT}.
SEQ ID NO: 22)
GCTAGCATGGATTACAAGGATGACGATGACAAGGGTGGATCTGGT
GGATCTGGTGGATCTGCCGACCGGGAAAAGCTGCTGACCGAGAGC
GGTGTTTACGGCACATTCGCTACATTTCAGATGGACCATGATTGG
TGGGACCTGCCTGGCGAATCCAGAGTGATCAGCGTGGCTGAAGTG
AAGGGCCTGGTCGAGCAGTGGAGCGGAAAGATCCTGGTGGAATCT
TATCTGCTGAGAGGCCTGAGCGACCACGCCGATCTGATGTTCAGA
GTGCACGCCAGAACCCTGTCTGATACCCAGCAGTTCCTGGCCGCC
TTTATGAACACCAGGCTGGGCAGACACCTGACCGACGGAGGACTT
CTGCACGGCGTGTCCAAGAAACCTACATACGTGGCCGGCTTCCCC
GAGTCTATGAAAACAGAGCTGCAGGTCAACGGCGAGAGCGGCAGC
AGACCTTACGCCATCGTGATTCCTATCAAGAAGGACGCCGAATGG
TGGATGCTGGACCAGGAGGCCAGAACAGCCCTGATGCAGGAGCAC
ACCCAGGCAGCTCTGCCATACCTGAAGACCGTGAAAAGAAAGCTG
TACCACAGCACCGGCCTGGACGACGTGGACTTCATCACCTACTTC
GAGACAGAGCGGCTGGAAGATTTTCACAACCTGGTGCGGGCCCTG
CAACAAGTGAAGGAGTTCAGACACAATCGGCGCTTCGGCCACCCT
ACCCTGCTGGGCACCATGAGCCCCCTGGATGAGATCCTCGAGAAG
TTCGCCCAGTGAGAATTC
AGC-S110A-GCC
GGC-G114N-AAC
AGC-S123D-GAC
GCC-A173M-ATG
SEQ ID NO: 23)
MDYKDDDDKGGSGGSGGSADREKLLTESGVYGTFATFQMDHDWWD
LPGESRVISVAEVKGLVEQWSGKILVESYLLRGLSDHADLMERVH
ARTLSDTQQFLAAFMNTRLGRHLTDGGLLHGVSKKPTYVAGFPES
MKTELQVNGESGSRPYAIVIPIKKDAEWWMLDQEARTALMQEHTQ
AALPYLKTVKRKLYHSTGLDDVDFITYFETERLEDFHNLVRALQQ
VKEFRHNRRFGHPTLLGTMSPLDEILEKFAQ*
FLAG-DaCld:
SEQ ID NO: 24)
GCTAGCATGGATTACAAGGATGACGATGACAAGGGTGGATCTGGT
GGATCTGGTGGATCTCAGCAGGCCATGCAGCCCATGCAGAGCATG
AAAATCGAGAGAGGAACCATCCTGACCCAGCCTGGCGTGTTCGGC
GTCTTTACCATGTTCAAGCTGCGCCCCGATTGGAACAAAGTGCCT
GTTGCTGAGAGAAAAGGCGCCGCTGAGGAAGTGAAAAAGCTGATC
GAGAAGCACAAGGACAACGTGCTGGTCGACCTTTATCTGACCAGA
GGCCTGGAAACCAACAGCGACTTTTTCTTCAGAATCAACGCCTAC
GACCTGGCCAAGGCCCAAACATTCATGAGAGAGTTCCGGAGCACC
ACCGTGGGCAAGAACGCCGATGTGTTTGAGACCCTGGTCGGCGTG
ACCAAGCCTCTGAATTACATCAGCAAGGATAAGTCCCCAGGCCTC
AACGCCGGCCTGTCTAGCGCTACATACAGCGGCCCTGCCCCTAGA
TACGTGATCGTGATTCCTGTGAAGAAAAATGCTGAATGGTGGAAT
ATGAGCCCCGAAGAGCGGCTGAAGGAGATGGAAGTGCACACAACC
CCTACCCTGGCCTACCTGGTGAACGTGAAGAGAAAGCTGTACCAC
AGCACTGGCCTGGACGACACCGACTTCATCACCTACTTCGAGACA
GATGACCTGACCGCCTTCAACAACCTGATGCTGTCTCTGGCCCAG
GTGAAGGAAAACAAGTTCCACGTGCGGTGGGGATCTCCAACAACA
CTGGGAACAATCCATTCTCCTGAGGACGTGATCAAGGCCCTGGCA
GATTGAGAATTC
SEQ ID NO: 5)
MDYKDDDDKGGSGGSGGSQQAMQPMQSMKIERGTILTQPGVFGVF
TMEKLRPDWNKVPVAERKGAAEEVKKLIEKHKDNVLVDLYLTRGL
ETNSDFFFRINAYDLAKAQTEMREFRSTTVGKNADVFETLVGVTK
PLNYISKDKSPGLNAGLSSATYSGPAPRYVIVIPVKKNAEWWNMS
PEERLKEMEVHTTPTLAYLVNVKRKLYHSTGLDDTDFITYFETDD
LTAFNNLMLSLAQVKENKFHVRWGSPTTLGTIHSPEDVIKALAD*
NwCld-FLAG:
SEQ ID NO: 26)
GCTAGCATGACTTTTACCGTGTTCACCGGCGGCGATAGCGGCGCC
TGGTCCATCCTGAGCGTGGCCCCAGTGATCGGCGAAAGCCTGATG
GCCGCTTCTCATCTGGCTATCGCCCCTAGCCTCAGCCTGGGCGAC
ACCAGCGCCACCACCCCTTGGCAACTGAGAGGCGTCGCCAGCCAC
GCCCGCTACGTGGAAAGAGCCGAGAAGATCGCCCTTACATCTGTG
CAGGCCGGCCTGGGAAGAAACGAGGCCACAAGAGCTGCTCTGATC
CCCATCAGAAAGTCCGCCGCCTGGTGGGAGATGACCCAGGACGAG
AGGCGGGCAATTTTCGAAGATAAGAGCCACCACATCGCTGCCAGC
CTGAAATACCTGCCTGCCATCGCCAGACAGCTGTATCACTGCAGA
GATATCGGAGAACCCTTTGACTTCCTGACATGGTTCGAGTACGCC
CCTGAGCACGCCACAATGTTCGAGGACCTGGTGGGCGTGCTGCGG
GCCACCGAGGAATGGACCTACGTTGAGCGGGAAGTGGACATCCGG
CTGGCCAGAGCCATCGGTGGATCTGGTGGATCTGGTGGATCTGAT
TACAAGGATGACGATGACAAGTAAGAATTC
SEQ ID NO: 27)
MTFTVFTGGDSGAWSILSVAPVIGESLMAASHLAIAPSLSLGDTS
ATTPWQLRGVASHARYVERAEKIALTSVQAGLGRNEATRAALIPI
RKSAAWWEMTQDERRAIFEDKSHHIAASLKYLPAIARQLYHCRDI
GEPFDELTWFEYAPEHATMFEDLVGVLRATEEWTYVEREVDIRLA
RAIGGSGGSGGSDYKDDDDK*
Mito-NwCld-FLAG:
SEQ ID NO: 28)
GCTAGCATGAGCGTGCTCACCCCACTCCTGCTGCGGGGGCTGACC
GGCAGCGCTACTTTTACCGTGTTCACCGGCGGCGATAGCGGCGCC
TGGTCCATCCTGAGCGTGGCCCCAGTGATCGGCGAAAGCCTGATG
GCCGCTTCTCATCTGGCTATCGCCCCTAGCCTCAGCCTGGGCGAC
ACCAGCGCCACCACCCCTTGGCAACTGAGAGGCGTCGCCAGCCAC
GCCCGCTACGTGGAAAGAGCCGAGAAGATCGCCCTTACATCTGTG
CAGGCCGGCCTGGGAAGAAACGAGGCCACAAGAGCTGCTCTGATC
CCCATCAGAAAGTCCGCCGCCTGGTGGGAGATGACCCAGGACGAG
AGGCGGGCAATTTTCGAAGATAAGAGCCACCACATCGCTGCCAGC
CTGAAATACCTGCCTGCCATCGCCAGACAGCTGTATCACTGCAGA
GATATCGGAGAACCCTTTGACTTCCTGACATGGTTCGAGTACGCC
CCTGAGCACGCCACAATGTTCGAGGACCTGGTGGGCGTGCTGCGG
GCCACCGAGGAATGGACCTACGTTGAGCGGGAAGTGGACATCCGG
CTGGCCAGAGCCATCGGTGGATCTGGTGGATCTGGTGGATCTGAT
TACAAGGATGACGATGACAAGTAAGAATTC
SEQ ID NO: 29)
MSVLTPLLLRGLTGSATFTVFTGGDSGAWSILSVAPVIGESLMAA
SHLAIAPSLSLGDTSATTPWQLRGVASHARYVERAEKIALTSVQA
GLGRNEATRAALIPIRKSAAWWEMTQDERRAIFEDKSHHIAASLK
YLPAIARQLYHCRDIGEPFDELTWFEYAPEHATMFEDLVGVLRAT
EEWTYVEREVDIRLARAIGGSGGSGGSDYKDDDDK*
HumanSLC5A5:
SEQ ID NO: 30)
GCTAGCATGGAAGCCGTGGAAACAGGCGAGAGACCTACATTCGGC
GCTTGGGATTACGGCGTCTTCGCCCTGATGCTGCTGGTGTCCACC
GGCATCGGCCTGTGGGGGGCCTGGCCAGAGGCGGCCAGCGGTCTG
CCGAGGACTTCTTCACCGGCGGCAGGCGGCTGGCCGCTCTGCCTG
TGGGCCTGAGCCTGAGCGCCAGCTTCATGTCTGCCGTTCAGGTAC
TGGGCGTTCCTTCTGAGGCCTACCGGTACGGCCTGAAGTTCCTGT
GGATGTGCCTGGGCCAGCTGCTGAACAGCGTGCTGACCGCCCTGC
TGTTCATGCCTGTGTTTTACAGACTGGGCCTGACAAGCACCTATG
AGTACCTGGAAATGAGATTCTCCAGGGCCGTGCGGCTGTGCGGCA
CCCTGCAATACATCGTGGCAACAATGCTGTACACCGGAATCGTCA
TTTACGCCCCTGCCCTGATCCTGAATCAGGTGACCGGACTGGATA
TCTGGGCCTCTCTGCTGAGCACAGGCATTATCTGCACCTTCTACA
CAGCCGTGGGCGGAATGAAAGCCGTGGTGTGGACCGATGTGTTCC
AGGTTGTGGTGATGCTGAGCGGGTTTTGGGTGGTCCTGGCCAGAG
GCGTGATGCTGGTCGGAGGGCCAAGACAGGTGCTGACCCTGGCTC
AGAACCACAGCAGAATCAACCTGATGGATTTCAACCCCGACCCCA
GAAGCAGATACACATTTTGGACCTTTGTGGTGGGAGGCACCCTGG
TGTGGCTGTCTATGTACGGAGTGAATCAAGCCCAGGTGCAGAGAT
ATGTGGCCTGCAGAACCGAGAAGCAGGCCAAGCTGGCCCTGCTCA
TCAACCAGGTGGGCCTTTTCCTGATCGTCAGCAGCGCCGCCTGCT
GCGGCATCGTGATGTTCGTGTTCTACACCGACTGCGACCCCCTGC
TCCTGGGCAGAATCTCCGCTCCAGACCAGTACATGCCCCTGCTGG
TGCTGGACATCTTCGAGGACCTGCCTGGCGTGCCTGGATTGTTTC
TGGCTTGTGCCTACAGCGGCACACTGAGCACCGCCAGCACCAGCA
TCAACGCCATGGCCGCCGTGACAGTGGAAGACCTGATTAAACCCC
GCCTGAGATCTCTGGCTCCTAGAAAGCTGGTTATCATCTCTAAGG
GCCTGAGCCTGATCTACGGCTCGGCGTGTCTGACCGTGGCCGCCT
TGAGCAGCCTGCTGGGAGGCGGCGTGCTGCAGGGCAGCTTCACCG
TGATGGGCGTGATCAGCGGCCCTCTGCTCGGAGCATTCATCCTGG
GCATGTTCCTGCCTGCCTGCAACACCCCTGGCGTACTCGCCGGCC
TGGGCGCTGGACTGGCCCTGAGCCTCTGGGTGGCCCTGGGCGCTA
CACTGTACCCCCCCAGCGAGCAGACCATGCGGGTGCTGCCATCCA
GCGCCGCACGGTGCGTGGCCTTGTCCGTGAACGCCTCTGGCCTCC
TGGATCCTGCTCTTCTGCCTGCCAATGATAGCTCCAGAGCCCCTA
GCAGCGGCATGGACGCCAGCAGGCCTGCCCTGGCTGATTCTTTCT
ATGCCATCAGCTACCTGTACTACGGCGCTCTGGGCACCCTGACCA
CCGTGCTTTGTGGCGCCCTGATCAGCTGCCTGACTGGGCCTACCA
AGCGGTCTACACTGGCCCCTGGACTGCTGTGGTGGGACCTGGCCC
GGCAGACAGCCAGCGTGGCCCCCAAGGAGGAAGTGGCTATCCTGG
ACGACAACCTGGTGAAGGGCCCGGAAGAGCTGCCCACCGGCAACA
AGAAACCTCCAGGCTTCCTCCCTACTAACGAGGACAGACTGTTTT
TCCTGGGACAAAAGGAACTGGAAGGCGCCGGCAGCTGGACACCTT
GTGTGGGCCACGACGGCGGAAGAGACCAGCAGGAGACGAACCTGT
GAGGTACC
SEQ ID NO: 31)
MEAVETGERPTFGAWDYGVFALMLLVSTGIGLWVGLARGGQRSAE
DFFTGGRRLAALPVGLSLSASFMSAVQVLGVPSEAYRYGLKFLWM
CLGQLLNSVLTALLEMPVFYRLGLTSTYEYLEMRFSRAVRLCGTL
QYIVATMLYTGIVIYAPALILNQVTGLDIWASLLSTGIICTFYTA
VGGMKAVVWTDVFQVVVMLSGFWVVLARGVMLVGGPRQVLTLAQN
HSRINLMDENPDPRSRYTFWTFVVGGTLVWLSMYGVNQAQVQRYV
ACRTEKQAKLALLINQVGLFLIVSSAACCGIVMFVFYTDCDPLLL
GRISAPDQYMPLLVLDIFEDLPGVPGLFLACAYSGTLSTASTSIN
AMAAVTVEDLIKPRLRSLAPRKLVIISKGLSLIYGSACLTVAALS
SLLGGGVLQGSFTVMGVISGPLLGAFILGMELPACNTPGVLAGLG
AGLALSLWVALGATLYPPSEQTMRVLPSSAARCVALSVNASGLLD
PALLPANDSSRAPSSGMDASRPALADSFYAISYLYYGALGTLTTV
LCGALISCLTGPTKRSTLAPGLLWWDLARQTASVAPKEEVAILDD
NLVKGPEELPTGNKKPPGELPTNEDRLFELGQKELEGAGSWTPCV
GHDGGRDQQETNL*
mito-FLAG-NdCld:
SEQ ID NO: 32)
GCTAGCATGCTCGCTACAAGGGTCTTTAGCCTCGTCGGAAAGAGA
GCTATCAGCACCTCCGTCTGCGTGAGAGCTCATGATTACAAGGAT
GACGATGACAAGGGTGGATCTGGTGGATCTGGTGGATCTGCCGAC
CGGGAAAAGCTGCTGACCGAGAGCGGTGTTTACGGCACATTCGCT
ACATTTCAGATGGACCATGATTGGTGGGACCTGCCTGGCGAATCC
AGAGTGATCAGCGTGGCTGAAGTGAAGGGCCTGGTCGAGCAGTGG
AGCGGAAAGATCCTGGTGGAATCTTATCTGCTGAGAGGCCTGAGC
GACCACGCCGATCTGATGTTCAGAGTGCACGCCAGAACCCTGTCT
GATACCCAGCAGTTCCTGAGCGCCTTTATGGGCACCAGGCTGGGC
AGACACCTGACCAGCGGAGGACTTCTGCACGGCGTGTCCAAGAAA
CCTACATACGTGGCCGGCTTCCCCGAGTCTATGAAAACAGAGCTG
CAGGTCAACGGCGAGAGCGGCAGCAGACCTTACGCCATCGTGATT
CCTATCAAGAAGGACGCCGAATGGTGGGCCCTGGACCAGGAGGCC
AGAACAGCCCTGATGCAGGAGCACACCCAGGCAGCTCTGCCATAC
CTGAAGACCGTGAAAAGAAAGCTGTACCACAGCACCGGCCTGGAC
GACGTGGACTTCATCACCTACTTCGAGACAGAGCGGCTGGAAGAT
TTTCACAACCTGGTGCGGGCCCTGCAACAAGTGAAGGAGTTCAGA
CACAATCGGCGCTTCGGCCACCCTACCCTGCTGGGCACCATGAGC
CCCCTGGATGAGATCCTCGAGAAGTTCGCCCAGTGAGAATTC
SEQ ID NO: 33)
MLATRVESLVGKRAISTSVCVRAHDYKDDDDKGGSGGSGGSADRE
KLLTESGVYGTFATFQMDHDWWDLPGESRVISVAEVKGLVEQWSG
KILVESYLLRGLSDHADLMERVHARTLSDTQQFLSAFMGTRLGRH
LTSGGLLHGVSKKPTYVAGFPESMKTELQVNGESGSRPYAIVIPI
KKDAEWWALDQEARTALMQEHTQAALPYLKTVKRKLYHSTGLDDV
DFITYFETERLEDFHNLVRALQQVKEFRHNRRFGHPTLLGTMSPL
DEILEKFAQ*

### REFERENCES

Raymond, J., and Segre, D. (2006). The effect of oxygen on biochemical networks and the evolution of complex life. Science 311, 1764-1767.
Lyons, T. W., Reinhard, C. T. and Planavsky, N. J. (2014). The rise of oxygen in Earth's early ocean and atmosphere. Nature 506, 307-315.
Nursall, J. R. (1959). Oxygen as a prerequisite to the origin of the metazoan. Nature 183, 1170-1172.
Zheng Lu and James A. Imlay. (2021). When anaerobes encounter oxygen: mechanisms of oxygen toxicity, tolerance and defence. Nature Reviews Microbiology 19, 774-785.
W G Kaelin, Jr. and P J Ratcliffe. (2008) Oxygen sensing by metazoans: the central role of the HIF hydroxylase pathway. Mol Cell 30, 393-402.
G L Semenza (2012) Hypoxia-inducible factors in physiology and medicine. Cell 148, 399-408.
M C Simon and B Keith (2008) The role of oxygen availability in embryonic development and stem cell function. Nat Rev Mol Cell Biol 9, 285-296.
Tslil Ast and Vamsi Mootha (2019). Oxygen and mammalian cell culture: are we repeating the experiment of Dr. Ox? Nature Metabolism 1(9):858-860.
Xiaokun Shu, Varda Lev-Ram, Thomas J. Deerinck, Yingchuan Qi, Ericka B. Ramko, Michael W. Davidson, Yishi Jin, Mark H. Ellisman, Roger Y. Tsien. 2011. A Genetically Encoded Tag for Correlated Light and Electron Microscopy of Intact Cells, Tissues, and Organisms. PLOS Biology 9(4), e1001041.
David G. Nicholls and Stuart J. Ferguson (2013). Bioenergetics 4. Academic Press.
Ettwig, K. F., Butler, M. K., Le Paslier, D., Pelletier, E., Mangenot, S., Kuypers, M. M. M., Schreiber, F., Dutilh, B. E., Zedelius, J., de Beer, D., Gloerich, J., Wessels, H. J. C. T., van Alen, T., Luesken, F., Wu, M. L., van de Pas-Schoonen, K. T., Op den Camp, H. J. M., Janssen-Megens, E. M., Francoijs, K.-J., Stunnenberg, H., Weissenbach, J., Jetten, M. S. M., and Strous, M. (2010). Nitrite-driven anaerobic methane oxidation by oxygenic bacteria. Nature 464(7288):543-8.
Stefan Hofbauer, Irene Schaffner, Paul G. Furtmüller and Christian Obinger. (2014) Chlorite dismutases—a heme enzyme family for use in bioremediation and generation of molecular oxygen. Biotechnology J. 9, 461-473
van Ginkel, C. G., Rikken, G. B., Kroon, A. G., Kengen, S. W. (1996). Purification and characterization of chlorite dismutase: a novel oxygen-generating enzyme. Arch. Microbiol. 166, 321-326.
Lee, A. Q., Streit, B. R., Zdilla, M. J., DuBois, J. L. (2008). Mechanism of and exquisite selectivity for O—O bond formation by the heme-dependent chlorite dismutase. Proceedings of the National Academy of Sciences U.S.A. 105(41):15654-9.
Laura M. K. Dassama, Timothy H. Yosca, Denise A. Conner, Michael H. Lee, Béatrice Blanc, Bennett R. Streit, Michael T. Green, Jennifer L. DuBois, Carsten Krebs, and J. Martin Bollinger Jr. (2012) O2-evolving Chlorite Dismutase as a Tool to Study O2-Utilizing Enzymes. Biochemistry 51(8):1607-1616.
Netzer et al., (2018) Ultrahigh specificity in a network of computationally designed protein-interaction pairs. Nature Comms. 9(1):5286.
Paul, K. G., Theorell, H., Åkeson, A. (1953). The molar light absorption of pyridine ferroprotoporphyrin (pyridine haemochromogen). Acta Chem Scand. 7:1284-1287.
Barr, I., Guo, F. (2015). Pyridine hemochromagen assay for determining the concentration of heme in purified protein solutions. Bio Protoc. 5(18):e1594.
Olp, M. D., Kalous, K. S., Smith, B. C. (2020). ICEKAT: an interactive online tool for calculating initial rates from continuous enzyme kinetic traces. BMC Bioinformatics. 21(1):186.
Hofbauer, S. et al. (2014). Transiently produced hypochlorite is responsible for the irreversible inhibition of chlorite dismutase. Biochemistry. 53(19):3145-57.
Kostan J., et al. (2010) Structural and functional characterization of the chlorite dismutase from the nitrite-oxidizing bacterium “Candidatus Nitrospira defluvii”: identification of a catalytically important amino acid residue. J Struct Biol 172(3):331-42.
Maixner, F., Wagner, M., Lucker, S., Pelletier, E., Schmitz-Esser, S., Hace, K., Spieck, E., Konrat, R., Le Paslier, D., Daims, H. (2008). Environmental genomics reveals a functional chlorite dismutase in the nitrite-oxidizing bacterium ‘Candidatus Nitrospira defluvii’. Environ. Microbiol. 10, 3043-3056.
Diana M Freire, Maria G Rivas, André M Dias, Ana T Lopes, Cristina Costa, Teresa Santos-Silva, Sabine Van Doorslaer, Pablo J. González (2015). The homopentameric chlorite dismutase from Magnetospirillum sp. J Inorg Biochem 151:1-9.
Bennet Streit and Jennifer L. DuBois (2008) Chemical and steady-state kinetic analyses of a heterologously expressed heme dependent chlorite dismutase. Biochemistry 47(19):5271-80.
Shaikh Nisar Ali and Riaz Mahmood (2017) Sodium chlorite increases production of reactive oxygen species that impair the antioxidant system and cause morphological changes in human erythrocytes. Environ Toxicol 32(4):1343-1353.
Magdalena Kwolek-Mirek, Grzegorz Bartosz, Corinne M. Spickett (2011). Sensitivity of antioxidant-deficient yeast to hypochlorite and chlorite. Yeast 28:595-609.
Sepehr Eskandari, Donald D. F. Loo, Ge Dai, Orlie Levy, Ernest M. Wright, and Nancy Carrasco (1997) Thyroid Na1/12 Symporter: Mechanism, Stoichiometry, and Specificity. Journal of Biological Chemistry 272(43):27230-27238.
T Sugano, N Oshino, and B Chance (1974) Mitochondrial functions under hypoxic conditions. The steady states of cytochrome c reduction and of energy metabolism. Biochim Biophys Acta 347, 340-358.
D V Titov et al., (2016) Complementation of mitochondrial electron transport chain by manipulation of the NAD+/NADH ratio. Science 352, 231-235.
V Cracan, D V Titov, H Shen, Z Grabarek, and V K Mootha (2017) A genetically encoded tool for manipulation of NADP(+)/NADPH in living cells. Nat Chem Biol 13, 1088-1095.
R P Goodman et al., (2020) Hepatic NADH reductive stress underlies common variation in metabolic traits. Nature 583, 122-126.
M E Harper and M E Patti (2020) Metabolic terminology: what's in a name? Nat Metab 2, 476-477.
Abdel-Rahman M S, Couri D, Bull RJ (1985) The kinetics of chlorite and chlorate in rats. Journal of Environmental Pathology, Toxicology and Oncology, 6(1): 97-103.
J. P. Bercz, L. Jones, L. Garner, D. Murray, D. A. Ludwig, and J. Boston (1982). Subchronic Toxicity of Chlorine Dioxide and Related Compounds in Drinking Water in the Nonhuman Primate. Environmental Health Perspectives 46:47-55.
Anna Schurich, Isabelle Magalhaes, Jonas Mattsson (2019). Metabolic regulation of CAR T cell function by the hypoxic microenvironment in solid tumors. Immunotherapy 11(4): 335-345.
John F. Mustard, S. L. Murchie, S. M. Pelkey, B. L. Ehlmann, R. E. Milliken, J. A. Grant, J.-P. Bibring, F. Poulet, J. Bishop, E. Noe Dobrea, L. Roach, F. Seelos, R. E. Arvidson, S. Wiseman, R. Green, C. Hash, D. Humm, E. Malaret, J. A. McGovern, K. Seelos, T. Clancy, R. Clark, D. Des Marais, N. Izenberg, A. Knudson, Y. Langevin, T. Martin (2008). Hydrated silicate minerals on Mars observed by the Mars Reconnaissance Orbiter CRISM instrument. Nature 454:305-309.
M H Hecht et al., (2009) Detection of perchlorate and the soluble chemistry of martian soil at the Phoenix lander site. Science 325, 64-67.
Olp, M. D., Kalous, K. S. & Smith, B. C. (2020) ICEKAT: an interactive online tool for calculating initial rates from continuous enzyme kinetic traces. BMC Bioinformatics 21, 186.
U.S. Pat. No. 10,724,010 to Coates, Recombinantly engineered cells expressing chlorite dismutase and methods for using same in cell culture

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated eukaryotic cell expressing a bacterial or archaeal chlorite: O2 lyase (Cld).

2. The isolated cell of claim 1, wherein the Cld is connected to a targeting sequence, optionally wherein the targeting sequence directs the Cld to the mitochondria.

3. The isolated cell of claim 1, wherein the Cld is expressed in the cytoplasm and/or the mitochondria.

4. The isolated cell of claim 1, which also expresses a chlorite transporter.

5. The isolated cell of claim 4, wherein the chlorite transporter is a sodium iodide symporter (NIS).

6. The isolated cell of claim 5, wherein the NIS is encoded by SLC5A5, optionally comprising a sequence shown in Table 1.

7. The isolated cell of claim 1, wherein the isolated eukaryotic cell is an animal cell.

8. The isolated cell of claim 7, wherein the animal is a mammalian cell.

9. The isolated cell of claim 8, wherein the mammalian cell is a human cell, optionally a CAR-T cell.

10. The isolated cell of claim 1, wherein the bacterial Cld is from Nitrospira defluvii (NdCld), Dechloromonas aromatica (DaCld), or Nitrobacter winogradskyi (NwCld).

11. The isolated cell of claim 1, wherein the bacterial or archaeal Cld lacks a functional periplasmic targeting sequence.

12. A method of generating oxygen in a eukaryotic cell, the method comprising culturing the cell of claim 1 in a media comprising 50 μm to 5 mM chlorite, or in at least 50, 70, 75, 100, 250, or 500 UM chlorite, or in up to 1, 2.5, or 5 mM chlorite.

13. The method of claim 12, wherein the cell is viable in media comprising at least 1, 2.5, or 5 mM chlorite.

14. A transgenic non-human uni- or multi-cellular eukaryotic organism comprising a cell of claim 1.

15. The transgenic non-human uni- or multi-cellular eukaryotic organism of claim 14, which is a worm or a mouse.

16. A method of generating oxygen in a transgenic non-human uni- or multi-cellular eukaryotic organism, the method comprising maintaining the organism of claim 14 in an environment comprising chlorite.

17. The method of claim 16, wherein the chlorite is present at levels that would be toxic to a non-transgenic organism of the same species.

18. An isolated Cld protein that lacks a functional periplasmic targeting sequence.

19. The isolated Cld protein of claim 18 further comprising a mitochondrial targeting sequence.

20. A nucleic acid comprising a sequence encoding the isolated Cld protein of claim 18, and optionally a sequence encoding a sodium iodide symporter (NIS).

21. The nucleic acid of claim 20, wherein the NIS is encoded by SLC5A5.

22. The nucleic acid of claim 20, wherein one or both of the sequences are codon optimized for expression in a eukaryotic cell, e.g., an animal cell, e.g., a human cell.

23. A vector comprising the nucleic acid of claim 20.

24. A host cell comprising the vector of claim 23.

25. The host cell of claim 24, which is an animal cell, e.g, a mammalian cell, e.g., a human cell.

26. The host cell of claim 24, wherein the bacterial Cld is from Nitrospira defluvii (NdCld), Dechloromonas aromatica (DaCld), or Nitrobacter winogradskyi (NwCld).

27. The host cell of claim 24, wherein the bacterial Cld lacks a functional periplasmic targeting sequence.

28. The host cell of claim 24, which also expresses a sodium iodide symporter (NIS).

29. The host cell of claim 28, wherein the NIS is encoded by SLC5A5, optionally comprising a sequence shown in Table 1.

30. A method of generating oxygen in a eukaryotic cell, the method comprising culturing the host cell of claim 24 in a media comprising 50 μm to 5 mM chlorite, or at least 50, 70, 75, 100, 250, or 500 uM chlorite, or up to 1, 2.5, or 5 mM chlorite.
